(19) United States
(12) Patent Application Publication
(10) Pub. No.: US 2024/0277758 A1
(43) Pub. Date: Aug. 22, 2024

(54) USE OF SODIUM THIOSULFATE FOR THE TREATMENT OF SULFUR MUSTARD EXPOSURE

(71) Applicant: Hope Medical Enterprises, Inc. DBA Hope Pharmaceuticals, Scottsdale, AZ (US)

(72) Inventors: Craig Sherman, Scottsdale, AZ (US); Micah Bodner, Eugene, OR (US); Jon Marshall, Eugene, OR (US)

(73) Assignee: Hope Medical Enterprises, Inc. dba Hope Pharmaceuticals, Scottsdale, AZ (US)

(21) Appl. No.: 18/397,569

(22) Filed: Dec. 27, 2023

Related U.S. Application Data
(60) Provisional application No. 63/435,670, filed on Dec. 28, 2022.

(51) Int. Cl. A61K 33/04; A61K 9/00; A61K 31/717

(52) U.S. Cl. CPC A61K 33/04; A61K 9/0014; A61K 31/717

(57) ABSTRACT

Provided herein are formulations comprising sodium thiosulfate. Also provided herein are methods of treating, preventing or ameliorating a sulfur mustard skin injury in a subject, wherein the methods comprise contacting the subject with a formulation provided herein.

### RELATED APPLICATIONS

This application claims the benefit of U.S. Provisional Application No. 63/435,670, filed Dec. 28, 2022, the disclosure of which is incorporated herein by reference in its entirety.

### FIELD

Provided herein are formulations comprising sodium thiosulfate. Also provided herein are methods of treating, preventing or ameliorating a sulfur mustard skin injury in a subject, wherein the methods comprise contacting the subject with a formulation comprising sodium thiosulfate provided herein.

### BACKGROUND

Sulfur mustard (HD, or “mustard”) is an extremely toxic chemical that was used as a chemical warfare agent during World War 1 between 1914 and 1918 and during the Iran-Iraq war between 1980 and 1988. The United States Food and Drug Administration (FDA) has not approved any drug product for the treatment of sulfur mustard-exposed victims. Given that only a small amount of sulfur mustard is necessary to potentially cause an enormous number of casualties on a battlefield or during a terrorist attack, the development of a safe and effective treatment of sulfur mustard-exposed victims would enhance public safety.

Sulfur mustard is an oily, lipophilic liquid that can penetrate skin and mucosal surfaces. Localized dermal lesions that can develop following prolonged skin contact with sulfur mustard include erythema, edema, vesication, and necrosis. Systemic toxicity such as decreased cellular respiration, inhibition of cellular reproduction, and cell death can occur throughout the body as sulfur mustard can penetrate through the skin and enter the blood stream.

It was recently reported that intravenous injection of sodium thiosulfate could be an effective antidote to sulfur mustard poisoning if administered before sulfur mustard exposure. However, pre-exposure antidotal treatment is impracticable with soldiers during war and impossible with civilians during a terrorist attack. It was also reported that injection of sodium thiosulfate after sulfur mustard exposure and systemic absorption provided no protection, and that the effectiveness of topical application of sodium thiosulfate was unknown. (McKinley et al., Letterman Army Institute of Research, Report No. 127, September 1982). There is a continuing need for treating, preventing or ameliorating a sulfur mustard skin injury that involves formulations and methods that are easy to administer and can minimize sulfur mustard dermal and systemic absorption.

### SUMMARY

Provided herein are formulations comprising sodium thiosulfate and a cellulose or a pharmaceutically acceptable salt thereof. In one embodiment, the formulations provided herein comprise sodium thiosulfate and methyl cellulose or a pharmaceutically acceptable salt thereof. In one embodiment, the formulations provided herein comprise sodium thiosulfate and carboxymethyl cellulose or a pharmaceutically acceptable salt thereof. In certain embodiments, the formulations further comprise a buffer. In one embodiment, the formulations provided herein are for topical administration.

In certain embodiments, provided herein are dosage forms comprising the formulations described herein, wherein the dosage forms are selected from the group consisting of creams, ointments, solutions, pastes, foams, liquid and aerosol sprays, films, gels, emulsions, microemulsions, lotions, eye rinse and nebulized mist for inhalation.

Also provided is a method of treating, preventing or ameliorating a sulfur mustard skin injury in a subject, wherein the method comprises contacting the subject with a therapeutically effective amount of a formulation provided herein. The therapeutically effective amount is effective to ameliorate or eliminate one or more symptoms of a sulfur mustard skin injury.

Also provided are methods of decontaminating a surface exposed to sulfur mustard comprising contacting the surface with a formulation provided herein.

Also provided are methods of decontaminating a surface exposed to nitrogen mustard comprising contacting the surface with a formulation provided herein.

In certain embodiments, provided herein are methods of preparing the formulations, wherein the methods comprise contacting sodium thiosulfate and a cellulose or a pharmaceutically acceptable salt thereof in an aqueous solvent.

Further provided herein are pharmaceutical packs or kits comprising one or more containers filled with one or more of the ingredients of the formulation. Optionally associated with such container(s) can be a notice in the form prescribed by a governmental agency regulating the manufacture, use or sale of pharmaceuticals or biological products, which notice reflects approval by the agency of manufacture, use of sale for human administration. The pack or kit can be labeled with information regarding mode of administration, sequence of drug administration (e.g., separately, sequentially or concurrently), or the like.

These and other aspects of the subject matter described herein will become evident upon reference to the following detailed description.

### BRIEF DESCRIPTION OF THE DRAWINGS

FIG. 1 provides a plot showing sulfur mustard hydrolytic half-life estimate.

FIG. 2 provides a plot showing overall kinetics of a reaction between sulfur mustard and an aqueous solution of sodium thiosulfate at a concentration of 250 milligram/milliliter.

FIG. 3 provides ¹H NMR spectra for a reaction of sulfur mustard with sodium octyl thiosulfate in 19:1 acetone-d6:D₂O, where A is ¹H NMR spectrum for sulfur mustard and B is ¹H NMR spectrum for sodium octyl thiosulfate.

FIG. 4 provides selected ¹H NMR spectra of the reaction of sulfur mustard with sodium octyl thiosulfate over 120 minutes.

FIG. 5 provides time course HPLC overlay of the reaction of sulfur mustard with sodium thiosulfate in 1:1 acetone:water. The scale for the plot is in ppm. HPLC trace A is at t=1-2 min; trace B is for t=10 min; trace C is for t=20 min; trace D is for t=30 min; trace E is for t=40 min; trace F is for t=50 min; and trace G is t=60 min.

FIG. 6 provides plots of time course of the reaction of sulfur mustard with different concentrations of sodium thiosulfate in 1/1 DMSO/water (0-60 min), analyzed by HPLC.

FIG. 7 provides selected spectra from ¹H NMR analysis of the reaction of sulfur mustard with sodium thiosulfate (10 eq) in 1/1 DMSO-d₆/D₂O. The scale for the spectra is in ppm.

FIG. 8 provides plots for half-life estimation for a reaction of sulfur mustard with sodium thiosulfate in 1/1 DMSO-d₆/D₂O using integration ratios from ¹H NMR.

FIG. 9 provides plots for degradation of sulfur mustard by sodium thiosulfate (50 eq.) in DMSO water mixtures, with different concentrations of sodium docedyl sulfate (SDS).

FIG. 10 provides plots for degradation of sulfur mustard by sodium thiosulfate (10 or 12.5 eq.) in DMSO water mixtures, with different concentrations of triton X-100.

FIG. 11 provides plots for degradation of sulfur mustard by sodium thiosulfate (50 eq.) in DMSO water mixtures, with different concentrations cetyl trimethylammonium carbonate (CATCO₃).

FIG. 12 provides an HPLC calibration curve for sulfur mustard (0.01-2.00 mg/mL).

FIG. 13 provides plots showing decomposition reaction rates for the reaction of sulfur mustard using n-decyl sodium sulfate as a modifier.

FIG. 14 provides plots showing decomposition reaction rates for the reaction of sulfur mustard using sodium lauryl sulfate as a modifier.

FIG. 15 provides plots showing decomposition reaction rates for the reaction of sulfur mustard using sodium lauryl sulfate as a modifier using various sodium thiosulfate concentrations.

FIG. 16 provides plots showing decomposition reaction rates for the reaction of sulfur mustard using triethanolamine as a modifier.

FIG. 17 provides a plot showing decomposition reaction rate for the reaction of sulfur mustard using tetrabutylammonium chloride as a modifier.

FIG. 18 provides plots showing decomposition reaction rates for the reaction of sulfur mustard using methyl cellulose as a modifier.

FIG. 19 provides plots showing decomposition reaction rates for the reaction of sulfur mustard using carboxymethyl cellulose as a modifier.

### DETAILED DESCRIPTION

### Definitions

The following detailed description is not to be taken in a limiting sense but is made merely for the purpose of illustrating the embodiments provided herein.

To facilitate understanding of the disclosure set forth herein, a number of terms are defined below.

Generally, the nomenclature used herein and the laboratory procedures in inorganic chemistry, analytical chemistry, organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. In the event that there is a plurality of definitions for a term used herein, those in this section prevail unless stated otherwise.

The term “subject” refers to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject.

As used herein, “treat,” “treating,” and “treatment” are meant to include a method of reducing, hindering, controlling, alleviating and/or reversing a condition and/or its attendant symptom(s) associated with an exposure to sulfur mustard in a subject.

The terms “prevent,” “preventing,” and “prevention” are meant to include a method of delaying and/or precluding the onset of a condition and/or its attendant symptom(s) associated with an exposure to sulfur mustard in the subject.

As used herein, amelioration of the symptoms of an exposure to sulfur mustard by administration of a particular formulation refers to any lessening, whether permanent or temporary, lasting or transient, that can be attributed to or associated with administration of the formulation.

The term “therapeutically effective amount” is meant to include the amount of a compound that, when administered, is sufficient to prevent development of, or alleviate to some extent, one or more of the symptoms of the condition being treated. The term “therapeutically effective amount” also refers to the amount of a compound that is sufficient to elicit the biological or medical response of a cell, tissue, system, animal, or human, which is being sought by a researcher, veterinarian, medical doctor, or clinician.

The term “composition” or “formulation” as used herein is intended to encompass a product comprising the specified ingredients (and in the specified amounts, if indicated), as well as any product which results, directly or indirectly, from combination of the specified ingredients in the specified amounts. Exemplary ingredients of a pharmaceutical formulation, suitable for use in contact with cells, tissues, or organs of humans and animals without excessive toxicity, irritation, allergic response, immunogenicity, or other problems or complications, commensurate with a reasonable benefit/risk ratio are known to one skill in the art. See, Remington: The Science and Practice of Pharmacy, 21st Edition, Lippincott Williams & Wilkins: Philadelphia, P A, 2005; Handbook of Pharmaceutical Excipients, 5th Edition, Rowe et al., Eds., The Pharmaceutical Press and the American Pharmaceutical Association: 2005; and Handbook of Pharmaceutical Additives, 3rd Edition, Ash and Ash Eds., Gower Publishing Company: 2007; Pharmaceutical Preformulation and Formulation, Gibson Ed., CRC Press LLC: Boca Raton, FL, 2004.

The terms “topical”, “topical (topical) dose” or “topical (topical) dosage form” and “transdermal”, “transdermal dose” or “transdermal formulation” as used herein include all forms (e.g., creams, ointments, solutions, pastes, foams, liquid and aerosol sprays, powders, films, gels, emulsions, microemulsions, and lotions) which are applied to the skin for a topical and/or systemic therapeutic effect.

The terms “active pharmaceutical ingredient”, “active ingredient” and “active substance” refer to a compound, which is administered, alone or in combination with one or more pharmaceutically acceptable excipients, to a subject for treating, preventing, or ameliorating one or more symptoms associated with an exposure to sulfur mustard. As used herein, “active pharmaceutical ingredient”, “active ingredient” and “active substance” may be an optically active isomer of a compound described herein. As used herein, “active pharmaceutical ingredient”, “active ingredient”, and “active substance” may be anhydrous, the monohydrate, dihydrate, trihydrate, quatrahydrate, pentahydrate, or other hydrated forms of sodium thiosulfate.

The term “sodium thiosulfate” includes anhydrous, monohydrate, dihydrate, trihydrate, quatrahydrate, pentahydrate, and other hydrated forms of sodium thiosulfate. In one embodiment, the “sodium thiosulfate” referred to herein is sodium thiosulfate pentahydrate (Na₂S₂O₃·5H₂O). In another embodiment, the sodium thiosulfate is pharmaceutical grade. The term “pharmaceutical grade” as used herein with respect to sodium thiosulfate means that the sodium thiosulfate was manufactured according to Good Manufacturing Practices (GMP) as detailed in the United States Code of Federal Regulations 21 CFR 211 and meets one or more of the purity levels recited herein.

The terms “non-purgeable organic carbon” (“NPOC”) and “non-volatile organic carbon” (“NVOC”) refer to organic carbon-based substances that are not volatile and are not purged from material when exposed to acid.

The term “about” or “approximately” means an acceptable error for a particular value as determined by one of ordinary skill in the art, which depends in part on how the value is measured or determined. In some embodiments, the term “about” or “approximately” means within 1, 2, 3, or 4 standard deviations. In some embodiments, the term “about” or “approximately” means within 15%, 10%, 9%, 8%, 7%, 6%, 5%, 4%, 3%, 2%, 1%, 0.5%, or 0.05% of a given value or range. In some embodiments, it is contemplated that the values preceded by the term “about” or “approximately” are exact.

### Sodium Thiosulfate

In some embodiments, the formulations provided herein comprise purified forms of sodium thiosulfate. In one embodiment, the formulations provided herein comprise pharmaceutical grade sodium thiosulfate. In another embodiment, the formulations provided herein comprise sodium thiosulfate meeting or exceeding one, more than one or all of the current United States Pharmacopoeia standards for sodium thiosulfate for pharmaceutical use. In another embodiment, the formulations provided herein comprise sodium thiosulfate that were manufactured according to Good Manufacturing Practices (GMP) as detailed in the United States Code of Federal Regulations 21 CFR 211. In one embodiment, the formulations provided herein comprise pharmaceutically acceptable sodium thiosulfate that is disclosed in U.S. Pat. Nos. 10,744,156 and 8,496,973, which are incorporated by reference herein in their entireties.

In one embodiment, the sodium thiosulfate is solid.

In one embodiment, the appearance of the sodium thiosulfate is colorless crystals.

In one embodiment, the appearance of a 10% solution containing the sodium thiosulfate is clear and colorless.

In one embodiment, the sodium thiosulfate is odorless.

In one embodiment, the presence of sodium in sodium thiosulfate used in the formulations herein is confirmed according to Method 191 in USP XXXII (2009), which is incorporated by reference herein in its entirety.

In one embodiment, the presence of thiosulfate in sodium thiosulfate used in the formulations herein is confirmed according to Method 191 in USP XXXII (2009).

In another embodiment, the sodium thiosulfate used in the formulations herein has a pH between about 6 to about 8 when measured in a 10% solution at 25° C. In some embodiments, the pH of the sodium thiosulfate provided herein is measured using a pH meter. In some embodiments, the pH of the sodium thiosulfate provided herein is determined according to Method 791 in USP XXXII (2009).

In yet another embodiment, the sodium thiosulfate used in the formulations herein has water content of about 32% to about 37% by weight. In some embodiments, the water content in the sodium thiosulfate used in the formulations herein is determined by Karl Fischer method. In some embodiments, the water content in the sodium thiosulfate used in the formulations herein is quantitated according to Method 921 in USP XXXII (2009).

In yet another embodiment, the heavy metal content in the sodium thiosulfate used in the formulations herein is no greater than about 10 ppm of a heavy metal. In yet another embodiment, the heavy metal content in the sodium thiosulfate used in the formulations herein is no greater than about 20 ppm of a heavy metal. The heavy metal content in the sodium thiosulfate used in the formulations herein is determined according to Method 231 in USP XXXII (2009).

In yet another embodiment, the sodium thiosulfate used in the formulations herein contains no greater than about 0.02% by weight of carbonate. In yet another embodiment, the sodium thiosulfate used in the formulations herein contains no greater than about 0.01% by weight of carbonate. In some embodiments, the amount of carbonate in the sodium thiosulfate used in the formulations herein is determined by contacting a sodium thiosulfate sample with an acid, such as phosphoric acid, to convert carbonate to carbon dioxide and determining the amount of the carbon dioxide using a non-dispersive infrared detector.

In yet another embodiment, the sodium thiosulfate used in the formulations herein contains no greater than about 0.005% by weight of insoluble matter. In some embodiments, the amount of insoluble material in the sodium thiosulfate used in the formulations herein is determined by dissolving 10 grams of the sodium thiosulfate used in the formulations herein in 100 mL of water, the solution is heated to boiling for 1 hour, the solution is filtered, washed with hot water, dried, cooled in a desiccator, and weighed.

In yet another embodiment, the sodium thiosulfate used in the formulations herein contains no greater than about 200 ppm by weight of chloride. In some embodiments, the chloride content in the sodium thiosulfate used in the formulations herein is determined according to Method 221 in USP XXXII (2009).

In yet another embodiment, the sodium thiosulfate used in the formulations herein contains no greater than about 0.002% by weight of iron. In some embodiments, the iron content in the sodium thiosulfate used in the formulations herein is determined using inductively coupled plasma mass spectrometry (ICP-MS). In some embodiments, the iron content in the sodium thiosulfate used in the formulations herein is determined using inductively coupled plasma-optical emission spectroscopy (ICP-OES). In some embodiments, the iron content in the sodium thiosulfate used in the formulations herein is determined according to Method 241 in USP XXXII (2009).

In yet another embodiment, the sodium thiosulfate used in the formulations herein contains no greater than about 0.001% by weight of lead. In some embodiments, the lead content in the sodium thiosulfate used in the formulations herein is determined according to Method 251 in USP XXXII (2009).

In yet another embodiment, the sodium thiosulfate used in the formulations herein contains no greater than about 0.01% by weight of calcium. In some embodiments, the calcium content in the sodium thiosulfate used in the formulations herein is determined using ICP-MS. In some embodiments, the calcium content in the sodium thiosulfate used in the formulations herein is determined using flame emission spectrometry (FES).

In yet another embodiment, the sodium thiosulfate used in the formulations herein causes no turbidity when ammonium oxalate test solution prepared according to USP XXXII (2009) is added to an aqueous solution containing sodium thiosulfate (e.g., one gram of sodium thiosulfate dissolved in 20 mL of water).

In yet another embodiment, the sodium thiosulfate used in the formulations herein contains no greater than about 0.005% by weight of potassium. In some embodiments, the potassium content in the sodium thiosulfate used in the formulations herein is determined using ICP-MS. In some embodiments, the potassium content in the sodium thiosulfate used in the formulations herein is determined using FES.

In yet another embodiment, the sodium thiosulfate used in the formulations herein contains no greater than about 0.05% by weight of sulfite, or no greater than about 0.1% by weight of sulfite. In some embodiments, the sulfite content in the sodium thiosulfate used in the formulations herein is determined according to the method for the determination of sulfite in American Chemical Society, Reagent Chemicals, 10^{th} Edition, incorporated by reference herein in its entirety.

In yet another embodiment, the sodium thiosulfate used in the formulations herein contains no greater than about 0.05%, no greater than about 0.1%, no greater than about 0.25%, or no greater than about 0.5% by weight of sulfate (as SO₄²⁻). In some embodiments, the sulfate content in the sodium thiosulfate used in the formulations herein is determined according to the method for the determination of sulfate in American Chemical Society, Reagent Chemicals, 10^{th} Edition.

In yet another embodiment, the sodium thiosulfate used in the formulations herein contains no greater than about 0.001% by weight of sulfide. In some embodiments, the sulfide content in the sodium thiosulfate used in the formulations herein is determined by the addition of lead (II) nitrate using methods described herein.

In yet another embodiment, the sodium thiosulfate used in the formulations herein contains no greater than about 0.002% by weight of nitrogen compounds (as N). In some embodiments, the nitrogen compounds (as N) content in the sodium thiosulfate used in the formulations herein is determined according to the method for the determination of nitrogen compounds in American Chemical Society, Reagent Chemicals, 10^{th} Edition.

In yet another embodiment, the sodium thiosulfate used in the formulations herein contains no greater than about 10 ppm, no greater than about 100 ppm, no greater than about 500 ppm, no greater than about 1000 ppm, or no greater than 5000 ppm of total volatile organic carbon. In some embodiments, the sodium thiosulfate used in the formulations herein contains no greater than the specific limits set forth in ICH Q3C(R3) for organic volatile impurities or a particular solvent (e.g., ethanol), the disclosure of which is incorporated by references in its entirety. In some embodiments, the content of organic volatile impurities is determined according to Method 467 in USP XXXII (2009).

In yet another embodiment, the sodium thiosulfate used in the formulations herein contains total NPOC of no greater than about 60 ppb, no greater than about 2.5 ppm, no greater than about 8 ppm, no greater than about 10 ppm, no greater than about 20 ppm, no greater than about 25 ppm, or no greater than about 50 ppm. In some embodiments, the sodium thiosulfate used in the formulations herein contains total NPOC of no greater than about 12 ppm. In some embodiments, the total NPOC in the sodium thiosulfate used in the formulations herein is determined using methods described herein. In some embodiments, the total NPOC in the sodium thiosulfate used in the formulations herein is determined by a) contacting the sodium thiosulfate with a predetermined amount of an inorganic acid-containing aqueous solution to form an aqueous sample solution; b) removing precipitates from the aqueous sample solution; c) contacting the sample solution with a predetermined amount of an oxidizer; and d) converting the organic carbon in the sample solution into carbon dioxide under a supercritical water oxidation (SCWO) condition.

In yet another embodiment, the sodium thiosulfate used in the formulations herein contains no greater than about 0.05 ppm of mercury. In some embodiments, the mercury content in the sodium thiosulfate used in the formulations herein is determined using ICP-MS. In some embodiments, the mercury content in the sodium thiosulfate used in the formulations herein is determined using ICP-OES. In some embodiments, the mercury content in the sodium thiosulfate used in the formulations herein is determined according to Method 261 in USP XXXII (2009).

In yet another embodiment, the sodium thiosulfate used in the formulations herein contains no greater than about 2 ppm of aluminum. In some embodiments, the aluminum content in the sodium thiosulfate used in the formulations herein is determined using ICP-MS. In some embodiments, the aluminum content in the sodium thiosulfate used in the formulations herein is determined using ICP-OES. In some embodiments, the aluminum content in the sodium thiosulfate used in the formulations herein is determined according to Method 206 in USP XXXII (2009).

In yet another embodiment, the sodium thiosulfate used in the formulations herein contains no greater than about 3 ppm of arsenic. In some embodiments, the arsenic content in the sodium thiosulfate used in the formulations herein is determined using ICP-MS. In some embodiments, the arsenic content in the sodium thiosulfate used in the formulations herein is determined using ICP-OES. In some embodiments, the arsenic content in the sodium thiosulfate used in the formulations herein is determined according to Method 211 in USP XXXII (2009).

In yet another embodiment, the sodium thiosulfate used in the formulations herein contains no greater than about 0.003% by weight of selenium. In some embodiments, the selenium content in the sodium thiosulfate used in the formulations herein is determined using ICP-MS. In some embodiments, the selenium content in the sodium thiosulfate used in the formulations herein is determined using ICP-OES. In some embodiments, the selenium content in the sodium thiosulfate used in the formulations herein is determined according to Method 291 in USP XXXII (2009).

In yet another embodiment, the total aerobic count of microbial load in the sodium thiosulfate used in the formulations herein is no greater than about 100 Colony Forming Units per gram (CFU/g). The total aerobic count of microbial load in the sodium thiosulfate used in the formulations herein is quantitated according to Method 61 in USP XXXII (2009).

In yet another embodiment, the total yeast and mold count in the sodium thiosulfate used in the formulations herein is no greater than about 20 CFU/g. The total yeast and mold count in the sodium thiosulfate used in the formulations herein is quantitated according to Method 61 in USP XXXII (2009).

In yet another embodiment, the sodium thiosulfate used in the formulations herein contains no greater than about 0.02 Endotoxin Units per milligram (EU/mg), no greater than about 0.1 EU/mg, or no greater than about 0.25 EU/mg of bacterial endotoxins. The amount of bacterial endotoxins in the sodium thiosulfate used in the formulations herein is quantitated according to Method 85 in USP XXXII (2009).

In yet another embodiment, the sodium thiosulfate used in the formulations herein contains no greater than 0.01% of a residual anti-caking agent.

In yet another embodiment, the sodium thiosulfate used in the formulations herein is characterized by one or more of the following:
containing no less than about 99% by weight and/or no greater than about 100.5% by weight of sodium thiosulfate on an anhydrous basis determined according to USP colorimetric assay;
containing no less than about 98% by weight and/or no greater than about 102% by weight of sodium thiosulfate on an anhydrous basis determined according to ion chromatography assay;
having a pH between about 6 to about 8 when measured in a 10% solution at 25° C.;
having water content of about 32% to about 37% by weight;
having an appearance of colorless crystals;
having a clear and colorless appearance as a 10% solution;
having no odor;
having a positive identification test for sodium;
having a positive identification test for thiosulfate;
having no turbidity when mixed with ammonium oxalate TS;
having heavy metal content of no greater than about 10 ppm;
having heavy metal content of no greater than about 20 ppm;
containing no greater than about 0.01% by weight of carbonate;
containing no greater than about 0.005% by weight of insoluble matter;
containing no greater than about 200 ppm of chloride;
containing no greater than about 0.001% by weight of sulfide;
containing no greater than about 0.05% or no greater than about 0.1% by weight of sulfite;
containing no greater than about 0.05%, no greater than about 0.1%,
no greater than about 0.25%, or no greater than about 0.5% by weight of sulfate;
containing no greater than about 0.002% by weight of iron;
containing no greater than about 0.01% by weight of calcium;
containing no greater than about 0.005% by weight of potassium;
containing no greater than about 10 ppm, no greater than about 100 ppm, no greater than about 500 ppm, no greater than about 1000 ppm, or no greater than 5000 ppm of organic volatile impurities;
having total NPOC of no greater than 60 ppb, no greater than about 2.5 ppm, no greater than about 8 ppm, no greater than about 10 ppm, no greater than about 20 ppm, no greater than about 25 ppm, or no greater than about 50 ppm;
containing no greater than about 0.05 ppm of mercury;
containing no greater than about 2 ppm of aluminum;
containing no greater than about 3 ppm of arsenic;
containing no greater than 0.001% by weight of lead;
containing no greater than about 0.002% by weight of nitrogen compounds (as N);
containing no greater than about 0.003% by weight of selenium;
containing no greater than 0.01% of a residual anti-caking agent;
having a total aerobic count of microbial load of no greater than about 100 CFU/g;
having a total yeast and mold count of no greater than about 20 CFU/g; and
containing no greater than about 0.02 EU/mg, no greater than about 0.1 EU/mg, or no greater than about 0.25 EU/mg of bacterial endotoxins.

In still another embodiment, the sodium thiosulfate used in the formulations herein is characterized by one or more of the following:
containing no less than about 99% by weight and/or no greater than about 100.5% by weight of sodium thiosulfate on an anhydrous basis determined according to USP colorimetric assay;
containing no less than about 98% by weight and/or no greater than about 102% by weight of sodium thiosulfate on an anhydrous basis determined according to ion chromatography assay;
having a pH between about 6 to about 8 when measured in a 10% solution at 25° C.;
having water content of about 32% to about 37% by weight;
having an appearance of colorless crystals;
having a clear and colorless appearance as a 10% solution;
having no odor;
having a positive identification test for sodium;
having a positive identification test for thiosulfate;
having no turbidity when mixed with ammonium oxalate TS;
having heavy metal content of no greater than about 10 ppm;
having heavy metal content of no greater than about 20 ppm;
containing no greater than about 0.01% by weight of carbonate;
containing no greater than about 0.005% by weight of insoluble matter;
containing no greater than about 200 ppm of chloride;
containing no greater than about 0.001% by weight of sulfide;
containing no greater than about 0.05% or no greater than about 0.1% by weight of sulfite;
containing no greater than about 0.05%, no greater than about 0.1%, no greater than about 0.25%, or no greater than about 0.5% by weight of sulfate;
containing no greater than about 0.002% by weight of iron;
containing no greater than about 0.01% by weight of calcium;
containing no greater than about 0.005% by weight of potassium;
containing no greater than about 10 ppm, no greater than about 100 ppm, no greater than about 500 ppm, no greater than about 1000 ppm, or no greater than 5000 ppm of organic volatile impurities;
having total NPOC of no greater than 60 ppb, no greater than about 2.5 ppm, no greater than about 8 ppm, no greater than about 10 ppm, no greater than about 20 ppm, no greater than about 25 ppm, or no greater than about 50 ppm;
containing no greater than about 0.05 ppm of mercury;
containing no greater than about 2 ppm of aluminum;
containing no greater than about 3 ppm of arsenic;
containing no greater than 0.001% by weight of lead;
containing no greater than about 0.002% by weight of nitrogen compounds (as N);
containing no greater than about 0.003% by weight of selenium;
having a total aerobic count of microbial load of no greater than about 100 CFU/g;
having a total yeast and mold count of no greater than about 20 CFU/g; and
containing no greater than about 0.02 EU/mg, no greater than about 0.1 EU/mg, or no greater than about 0.25 EU/mg of bacterial endotoxins.

In yet another embodiment, the sodium thiosulfate used in the formulations herein is characterized by one or more of the following:
containing no less than about 99% by weight and/or no greater than about 100.5% by weight of sodium thiosulfate on an anhydrous basis determined according to USP colorimetric assay;
having water content of about 32% to about 37% by weight; and
having heavy metal content of no greater than about 20 ppm.

In some embodiments, where the sodium thiosulfate is described as “containing no greater than” a certain amount of a particular material, the sodium thiosulfate does not contain a detectable amount of the material.

### Exemplary Formulations

Provided herein are formulations comprising sodium thiosulfate, and a cellulose or a pharmaceutically acceptable salt thereof.

Provided herein are formulations comprising sodium thiosulfate, and a cellulose or a pharmaceutically acceptable salt thereof in a pharmaceutically acceptable carrier.

In certain embodiments, the formulations provided herein comprise about 0.1% to about 25% sodium thiosulfate based on total weight of the formulation.

In certain embodiments, the formulations provided herein comprise about 1% to about 25% sodium thiosulfate based on total weight of the formulation. In certain embodiments, the formulations provided herein comprise about 1% to about 20% sodium thiosulfate based on total weight of the formulation. In certain embodiments, the formulations provided herein comprise about 2% to about 15% sodium thiosulfate based on total weight of the formulation. In certain embodiments, the formulations provided herein comprise about 5% to about 15% sodium thiosulfate based on total weight of the formulation. In certain embodiments, the formulations provided herein comprise about 2% to about 10% sodium thiosulfate based on total weight of the formulation. In certain embodiments, the formulations provided herein comprise about 1%, 2%, 5%, 7%, 10%, 12%, 15%, 17%, 20% or 25% sodium thiosulfate based on total weight of the formulation.

In certain embodiments, the cellulose for use in the formulations provided herein is selected from the group consisting of methyl cellulose, ethyl cellulose, cellulose acetate, carboxymethyl cellulose, sodium salt of carboxymethyl cellulose (carboxymethyl cellulose sodium), hydroxyethylcellulose (HEC), hydroxypropylcellulose (HPC), hydroxypropyl methyl cellulose (HPMC), microcrystalline celluloses, such as AVICEL-PH-101, AVICEL-PH-103, AVICEL RC-581, AVICEL-PH-105 (FMC Corp., Marcus Hook, PA), and mixtures thereof. In certain embodiments, the cellulose for use in the formulations provided herein is selected from the groups consisting of methyl cellulose, carboxymethyl cellulose, carboxymethyl cellulose sodium, and a mixture thereof. In certain embodiments, the cellulose for use in the formulations provided herein is carboxymethyl cellulose or carboxymethyl cellulose sodium.

In certain embodiments, the formulations provided herein comprise about 0.01% to about 5% cellulose or a pharmaceutically acceptable salt thereof, based on total weight of the formulation. In certain embodiments, the formulations provided herein comprise about 0.05% to about 3% cellulose or a pharmaceutically acceptable salt thereof, based on total weight of the formulation. In certain embodiments, the formulations provided herein comprise about 0.1% to about 2% cellulose or a pharmaceutically acceptable salt thereof, based on total weight of the formulation. In certain embodiments, the formulations provided herein comprise about 0.5% to about 2% cellulose or a pharmaceutically acceptable salt thereof, based on total weight of the formulation. In certain embodiments, the formulations provided herein comprise about 1% cellulose or a pharmaceutically acceptable salt thereof, based on total weight of the formulation. In certain embodiments, the formulations provided herein comprise about 0.01%, 0.05%, 0.1%, 0.5%, 1%, 2%, 3%. 4% or 5% cellulose or a pharmaceutically acceptable salt thereof, based on total weight of the formulation.

In certain embodiments, the formulations provided herein comprise about 0.01% to about 5% carboxymethyl cellulose or a pharmaceutically acceptable salt thereof, based on total weight of the formulation. In certain embodiments, the formulations provided herein comprise about 0.05% to about 3% carboxymethyl cellulose or a pharmaceutically acceptable salt thereof, based on total weight of the formulation. In certain embodiments, the formulations provided herein comprise about 0.1% to about 2% carboxymethyl cellulose or a pharmaceutically acceptable salt thereof, based on total weight of the formulation. In certain embodiments, the formulations provided herein comprise about 0.5% to about 2% carboxymethyl cellulose or a pharmaceutically acceptable salt thereof, based on total weight of the formulation. In certain embodiments, the formulations provided herein comprise about 1% carboxymethyl cellulose or a pharmaceutically acceptable salt thereof, based on total weight of the formulation. In certain embodiments, the formulations provided herein comprise about 0.01%, 0.05%, 0.1%, 0.5%, 1%, 2%, 3%. 4% or 5% carboxymethyl cellulose or a pharmaceutically acceptable salt thereof, based on total weight of the formulation.

In certain embodiments, the formulations provided herein comprise about 0.01% to about 5% carboxymethyl cellulose sodium salt, based on total weight of the formulation. In certain embodiments, the formulations provided herein comprise about 0.05% to about 3% carboxymethyl cellulose sodium salt, based on total weight of the formulation. In certain embodiments, the formulations provided herein comprise about 0.1% to about 2% carboxymethyl cellulose sodium salt, based on total weight of the formulation. In certain embodiments, the formulations provided herein comprise about 0.5% to about 2% carboxymethyl cellulose sodium salt, based on total weight of the formulation. In certain embodiments, the formulations provided herein comprise about 1% carboxymethyl cellulose sodium salt, based on total weight of the formulation. In certain embodiments, the formulations provided herein comprise about 0.01%, 0.05%, 0.1%, 0.5%, 1%, 2%, 3%. 4% or 5% carboxymethyl cellulose sodium salt, based on total weight of the formulation.

In certain embodiments, the formulations provided herein further comprise a buffer to control pH. Suitable buffering agents for use in the formulations provided herein include, but are not limited to, an acetate buffer, a borate buffer, a carbonate buffer, a citrate buffer, a phosphate buffer, and a sulfate buffer. In certain embodiments, the formulations provided herein comprise a borate buffer. In certain embodiments, a borate buffer includes boric acid.

In certain embodiments, the formulations provided herein further comprise one or more pharmaceutically or dermatologically acceptable excipients, adjuvants or carriers. Nonlimiting examples of these include solvents, emulsifiers, suspending agents, decomposers, binding agents, stabilizing agents, diluents, gelling agents, emollients, thickening agent, preservatives, lubricants, and the like, combinations thereof, or the like. Exemplary excipients, adjuvants or carriers are disclosed in U.S. Patent Publication No. 2017/0333410, 2022/0023174, and 2009/0048153.

In one embodiment, the pharmaceutically or dermatologically acceptable excipients, adjuvants or carriers including lubricants, such as a natural or synthetic fat or oil, e.g., a tris-fatty acid glycerate or lecithin.

In one embodiment, the pharmaceutically or dermatologically acceptable excipients, adjuvants or carriers including emollients such as hydrocarbon oils and waxes, silicone oils, vegetable, animal or marine fats or oils, glyceride derivatives, fatty acids or fatty acid esters, lanolin and derivatives, wax esters, sterols, phospholipids and the like. The fatty acid esters may be mono- or di-esters. Exemplary fatty di-esters include dibutyl adipate, diethyl sebacate, diisopropyl dimerate, and dioctyl succinate. Exemplary branched chain fatty esters include 2-ethyl-hexyl myristate, isopropyl stearate and isostearyl palmitate. Exemplary tribasic acid esters include triisopropyl trilinoleate and trilauryl citrate. Exemplary straight chain fatty esters include lauryl palmitate, myristyl lactate, oleyl eurcate and stearyl oleate. Exemplary fatty acids include those compounds having from 10 to 20 carbon atoms, such as cetyl, arachidyl, behenyl, cetearyl, myristyl, palmitic and stearyl acids. Exemplary hydrocarbons which may serve as emollients are those having hydrocarbon chains anywhere from 12 to 30 carbon atoms. Exemplary hydrocarbons include mineral oil, petroleum jelly, paraffin oil, squalene and isoparaffins.

In one embodiment, the pharmaceutically or dermatologically acceptable excipients, adjuvants or carriers include, thickeners, such as carboxylic acid polymers, crosslinked polyacrylate polymers, crosslinked vinyl ether/maleic anhydride copolymers, crosslinked poly (N-vinylpyrrolidone), polyacrylamide polymers, polysaccharides, and gums including xanthan, carrageenan, gelatin, karaya, pectin and locust beans gum.

In one embodiment, the pharmaceutically or dermatologically acceptable excipients, adjuvants or carriers include emulsifiers, including but not limited to sodium docedyl sulfate (SDS), PEG1500, alfa-cyclodextrin, tetrabutylammonium bromide, cetyltrimethylammonium carbonate, decyl polyglucoside, lauryl polyglucoside, ceteth-6, ceteth-10, ceteth-12, steareth-6, steareth-10, steareth-12, PEG-6 stearate, PEG-10 stearate, PEG-100 stearate, PEG-12 stearate, PEG-20 glyceryl stearate, PEG-80 glyceryl tallowate, PEG-10 glyceryl stearate, PEG-30 glyceryl cocoate, PEG-80 glyceryl cocoate. PEG-200 glyceryl tallowate, PEG-8 dilaurate, PEG-10 distearate, steareth-21, ceteareth-20, ceteareth-12, sucrose cocoate, steareth-100, PEG-100 stearate, polyethylene glycol 20 sorbitan monolaurate (polysorbate 20), polyethylene glycol 5 soya sterol, steareth-20, ceteareth-20, PPG-2 methyl glucose ether distearate, ceteth-10, polysorbate 80, cetyl phosphate, potassium cetyl phosphate, cetyl diethanolamine phosphate, polysorbate 60, glyceryl stearate, polyoxyethylene 20 sorbitan trioleate (polysorbate 85), sorbitan monolaurate, polyoxyethylene 4 sodium laureth stearate, polyglyceryl-4 isostearate, hexyl laurate. PPG-2 methyl glucose ether distearate Stearate, PEG-100 stearate, sorbitan stearate and sucrose cocoate. Additional emulsifiers are described for example, in Allured, M.2009 McCutcheon's Emulsifiers and Detergents, McCutcheon's Publications, Apr. 1, 2009 and U.S. Pat. No. 5,968,528. In one embodiment, the emulsifier is sodium docedyl sulfate (SDS), PEG1500, alfa-cyclodextrin, tetrabutylammonium bromide or cetyltrimethylammonium carbonate.

In one embodiment, the pharmaceutically or dermatologically acceptable excipients, adjuvants or carriers include surfactants, including but not limited to sodium laureth sulfate, sodium laureth-13 carboxylate, disodium laureth sulfosuccinate, disodium cocoamphodiacetate, glycol stearate, PEG-150 distearate and the like, and mixtures thereof. In one embodiment, the surfactant is selected from the group consisting of lauroamphocarboxypropionate, lauroamphopropionate, lauroamphoglycinate, lauroamphocarboxyglycinate, lauroamphopropylsulfonate, lauroamphocarboxypropionic acid, myristoamphocarboxy-propionate, myristoamphopropionate, myristoamphoglycinate, myristoamphocarboxyglycinate, myristoamphopropylsulfonate, myristoamphocarboxypropionic acid, cocoamphocarboxypropionate, cocoamphopropionate, cocoamphoglycinate, cocoamphocarboxyglycinate, cocoamphopropylsulfonate, cocoamphocarboxypropionic acid and mixtures thereof.

The formulations may contain any pharmaceutically or dermatologically acceptable excipients, adjuvants or carriers in any desired amount, having any desired physicochemical form or mechanical properties (e.g., viscosity, spreadability).

In certain embodiments, the formulations provided herein comprise about 0.1% to about 25% sodium thiosulfate and about 0.01% to about 5% carboxymethyl cellulose or a pharmaceutically acceptable salt thereof, based on total weight of the formulation.

In certain embodiments, the formulations provided herein comprise about 0.1% to about 25% sodium thiosulfate and about 0.01% to about 5% carboxymethyl cellulose or a pharmaceutically acceptable salt thereof, based on total weight of the formulation and a buffer.

In certain embodiments, the formulations provided herein comprise about 1% to about 20% sodium thiosulfate and about 0.1% to about 2% carboxymethyl cellulose or a pharmaceutically acceptable salt thereof, based on total weight of the formulation and a buffer.

In certain embodiments, the formulations provided herein comprise about 1% to about 20% sodium thiosulfate and about 0.1% to about 2% carboxymethyl cellulose sodium salt, based on total weight of the formulation and a buffer.

In certain embodiments, the formulations provided herein comprise about 1% to about 20% sodium thiosulfate and about 0.1% to about 2% carboxymethyl cellulose sodium salt, based on total weight of the formulation and a borate buffer.

In certain embodiments, the formulations provided herein comprise about 1% to about 20% sodium thiosulfate and about 0.1% to about 2% carboxymethyl cellulose sodium salt, based on total weight of the formulation and a 50 mM borate buffer.

In certain embodiments, the formulations provided herein comprise about 2% to about 10% sodium thiosulfate and about 1% to about 2% carboxymethyl cellulose sodium salt, based on total weight of the formulation and a borate buffer.

In certain embodiments, the formulations provided herein comprise about 2% to about 10% sodium thiosulfate and about 0.1% to about 2% carboxymethyl cellulose sodium salt, based on total weight of the formulation and a borate buffer.

In certain embodiments, the formulations provided herein comprise about 10% sodium thiosulfate and about 1% carboxymethyl cellulose sodium salt, based on total weight of the formulation and a borate buffer.

In certain embodiments, the formulations provided herein comprise about 10% sodium thiosulfate and about 1% carboxymethyl cellulose sodium salt, based on total weight of the formulation and a 50 mM borate buffer.

In one embodiment, the formulations provided herein comprise sodium thiosulfate and one or more of n-decyl sodium sulfate, sodium lauryl sulfate, triethanolamine, tetrabutylammonium chloride, methyl cellulose and carboxymethyl cellulose sodium. In one embodiment, the formulations provided herein comprise sodium thiosulfate and n-decyl sodium sulfate. In one embodiment, the formulations provided herein comprise sodium thiosulfate and sodium lauryl sulfate. In one embodiment, the formulations provided herein comprise sodium thiosulfate and triethanolamine. In one embodiment, the formulations provided herein comprise sodium thiosulfate tetrabutylammonium chloride. In one embodiment, the formulations provided herein comprise sodium thiosulfate and methyl cellulose.

### Dosage Forms

In certain embodiments, provided herein are dosage forms comprising the formulations described herein, wherein the dosage forms are selected from the group consisting of creams, ointments, solutions, pastes, foams, liquid and aerosol sprays, films, gels, emulsions, microemulsions, lotions, eye rinse and nebulized mist for inhalation. Exemplary dosage forms are disclosed in, e.g., Williams AC. Transdermal and Topical Drug Delivery, Pharmaceutical Press, London, 2003; Remington: The Science and Practice of Pharmacy, 21^{st} edition, Lippincott Williams & Wilkins, Baltimore, 2005; Watters KA. Dermatological and Transdermal Formulations, information Healthcare, 1^{st} edition, 2002; and Transdermal Drug Delivery, Hargrove J (eds.), 2^{nd} edition, 2002.

The formulations provided herein can be packaged in a suitable container to suit its viscosity and intended use. In certain embodiments, a formulation is a lotion or fluid cream that can be packaged in a bottle, a propellant-driven aerosol device or a container fitted with a pump suitable for finger operation. In certain embodiments, a formulation is a cream that can be stored in a non-deformable bottle or squeeze container, such as a tube or a lidded jar.

Further provided herein are pharmaceutical packs or kits comprising one or more containers filled with one or more of the ingredients of the formulation. In one embodiment, the kit comprises a notice in the form prescribed by a governmental agency regulating the manufacture, use or sale of pharmaceuticals or biological products, wherein the notice reflects approval by the agency of manufacture, use of sale for human administration. The pack or kit can be labeled with information regarding mode of administration, sequence of drug administration (e.g., separately, sequentially or concurrently), or the like. In one embodiment, the kit further comprises an absorbent applicator for applying the formulation to the subject exposed with sulfur mustard. In one embodiment, the absorbent applicator includes an absorbent sponge, woven or non-woven fabric, cloth, paper towel or towelette. In another embodiment, the applicator is saturated with the formulation prior to packaging. In another embodiment, the kit further includes a container for safe disposal of used applicators.

### Processes for Making Formulations and Formulation Prepared by the Processes

The formulations provided herein can be prepared by any of the methods known in the art and as described herein. In certain embodiments, provided herein are methods for preparing a formulation by contacting sodium thiosulfate with a cellulose or a pharmaceutically acceptable salt thereof to obtain the formulation.

In certain embodiments, the methods provided herein comprise contacting sodium thiosulfate with a cellulose or a pharmaceutically acceptable salt thereof in a pharmaceutically acceptable excipient. One aspect of the methods provided herein comprises contacting a monohydrate, dihydrate, trihydrate, quatrahydrate, pentahydrate, or other hydrated forms of sodium thiosulfate, with a cellulose or a pharmaceutically acceptable salt thereof in a pharmaceutically acceptable excipient. One aspect of the methods provided herein comprises contacting a pentahydrate form of sodium thiosulfate with a cellulose or a pharmaceutically acceptable salt thereof in a pharmaceutically acceptable excipient. One aspect of the methods provided herein comprises contacting an anhydrous sodium thiosulfate with a cellulose or a pharmaceutically acceptable salt thereof in a pharmaceutically acceptable excipient. One aspect of the methods provided comprises contacting a pharmaceutical grade sodium thiosulfate with a cellulose or a pharmaceutically acceptable salt thereof in a pharmaceutically acceptable excipient. One aspect of the methods provided herein comprises contacting a purified form of sodium thiosulfate with a cellulose or a pharmaceutically acceptable salt thereof in a pharmaceutically acceptable excipient. One aspect of the methods provided herein comprises contacting a sodium thiosulfate with a cellulose or a pharmaceutically acceptable salt thereof in a pharmaceutically acceptable excipient, wherein the sodium thiosulfate meets or exceeds one, more than one or all of the current United States Pharmacopoeia standards for sodium thiosulfate for pharmaceutical use.

In certain embodiments, provided herein are formulations prepared by contacting a monohydrate, dihydrate, trihydrate, quatrahydrate, pentahydrate, or other hydrated forms of sodium thiosulfate with a cellulose or a pharmaceutically acceptable salt thereof in a pharmaceutically acceptable excipient. In one embodiment, provided herein are formulations prepared by contacting a pentahydrate sodium thiosulfate with a cellulose or a pharmaceutically acceptable salt thereof in a pharmaceutically acceptable excipient. In one embodiment, provided herein are formulations prepared by contacting an anhydrous sodium thiosulfate with a cellulose or a pharmaceutically acceptable salt thereof in a pharmaceutically acceptable excipient. In one embodiment, provided herein are formulations prepared by contacting a pharmaceutical grade sodium thiosulfate with a cellulose or a pharmaceutically acceptable salt thereof in a pharmaceutically acceptable excipient. One aspect of the formulations provided herein are prepared by contacting a purified form of sodium thiosulfate with a cellulose or a pharmaceutically acceptable salt thereof in a pharmaceutically acceptable excipient. One aspect of the formulations provided herein are prepared by contacting a sodium thiosulfate with a cellulose or a pharmaceutically acceptable salt thereof in a pharmaceutically acceptable excipient, wherein the sodium thiosulfate meets or exceeds one, more than one or all of the current United States Pharmacopoeia standards for sodium thiosulfate for pharmaceutical use.

In one embodiment, the methods provided herein comprise contacting sodium thiosulfate with a cellulose or a pharmaceutically acceptable salt thereof in an aqueous solution. In one embodiment, the methods provided herein comprise contacting sodium thiosulfate with a cellulose or a pharmaceutically acceptable salt thereof in a buffer. In one embodiment, the buffer is selected from a group consisting of an acetate buffer, a borate buffer, a carbonate buffer, a citrate buffer, a phosphate buffer, and a sulfate buffer. In one embodiment, the methods provided herein comprise contacting sodium thiosulfate with a cellulose or a pharmaceutically acceptable salt thereof in a borate buffer.

In one embodiment, the methods provided herein comprise contacting sodium thiosulfate with carboxymethyl cellulose or a pharmaceutically acceptable salt thereof in a pharmaceutically acceptable excipient. In one embodiment, the methods provided herein comprise contacting sodium thiosulfate with carboxymethyl cellulose or a pharmaceutically acceptable salt thereof in a buffer. In one embodiment, the methods provided herein comprise contacting sodium thiosulfate with carboxymethyl cellulose or a pharmaceutically acceptable salt thereof in a borate buffer. In one embodiment, the methods provided herein comprise contacting sodium thiosulfate with carboxymethyl cellulose or a pharmaceutically acceptable salt thereof in a 50 mM borate buffer.

In one embodiment, the methods provided herein comprise contacting about 0.1% to about 25% sodium thiosulfate and about 0.01% to about 5% carboxymethyl cellulose or a pharmaceutically acceptable salt thereof, based on total weight of the formulation. In one embodiment, the methods provided herein comprise contacting about 0.1% to about 25% sodium thiosulfate and about 0.01% to about 5% carboxymethyl cellulose or a pharmaceutically acceptable salt thereof, based on total weight of the formulation, in a pharmaceutically acceptable carrier. In one embodiment, the methods provided herein comprise contacting about 0.1% to about 25% sodium thiosulfate and about 0.01% to about 5% carboxymethyl cellulose or a pharmaceutically acceptable salt thereof, based on total weight of the formulation, in a buffer. In one embodiment, the methods provided herein comprise contacting about 0.1% to about 25% sodium thiosulfate and about 0.01% to about 5% carboxymethyl cellulose or a pharmaceutically acceptable salt thereof, based on total weight of the formulation, in a borate buffer. In one embodiment, the methods provided herein comprise contacting about 0.1% to about 25% sodium thiosulfate and about 0.01% to about 5% carboxymethyl cellulose or a pharmaceutically acceptable salt thereof, based on total weight of the formulation, in a 50 mM borate buffer.

In one embodiment, the methods provided herein comprise contacting about 0.1% to about 25% sodium thiosulfate and about 0.01% to about 5% carboxymethyl cellulose sodium salt based on total weight of the formulation. In one embodiment, the methods provided herein comprise contacting about 0.1% to about 25% sodium thiosulfate and about 0.01% to about 5% carboxymethyl cellulose sodium salt based on total weight of the formulation in a pharmaceutically acceptable carrier. In one embodiment, the methods provided herein comprise contacting about 0.1% to about 25% sodium thiosulfate and about 0.01% to about 5% carboxymethyl cellulose sodium salt based on total weight of the formulation, in a buffer. In one embodiment, the methods provided herein comprise contacting about 0.1% to about 25% sodium thiosulfate and about 0.01% to about 5% carboxymethyl cellulose sodium salt based on total weight of the formulation, in a borate buffer. In one embodiment, the methods provided herein comprise contacting about 0.1% to about 25% sodium thiosulfate and about 0.01% to about 5% carboxymethyl cellulose sodium salt based on total weight of the formulation, in a 50 mM borate buffer.

In one embodiment, the methods provided herein comprise contacting sodium thiosulfate with a cellulose or a pharmaceutically acceptable salt thereof in a buffer to obtain a solution, and further contacting the solution with an excipient selected from an emulsifier, a suspending agent, a decomposer, a binding agent, a stabilizing agent, a surfactant, a diluent, a gelling agent, an emollient, a thickening agent, a preservative and a lubricant, or a combination thereof.

In one embodiment, the methods provided herein comprise contacting sodium thiosulfate with carboxymethyl cellulose or a pharmaceutically acceptable salt thereof in a buffer to obtain a solution, and further contacting the solution with an excipient selected from an emulsifier, a suspending agent, a decomposer, a binding agent, a stabilizing agent, a surfactant, a diluent, a gelling agent, an emollient, a thickening agent, a preservative and a lubricant, or a combination thereof.

In one embodiment, the methods provided herein comprise contacting sodium thiosulfate with carboxymethyl cellulose sodium salt in a buffer to obtain a solution, and contacting the solution with an excipient selected from an emulsifier, a suspending agent, a decomposer, a binding agent, a stabilizing agent, a surfactant, a diluent, a gelling agent, an emollient, a thickening agent, a preservative and a lubricant, or a combination thereof.

In one embodiment, the methods provided herein comprise contacting sodium thiosulfate with carboxymethyl cellulose sodium salt in a borate buffer to obtain a solution, and contacting the solution with an excipient selected from an emulsifier, a suspending agent, a decomposer, a binding agent, a stabilizing agent, a surfactant, a diluent, a gelling agent, an emollient, a thickening agent, a preservative and a lubricant, or a combination thereof.

### Methods of Use

Provided herein are methods for treating, preventing or ameliorating a sulfur mustard skin injury in a subject, wherein the methods comprise contacting the subject with a therapeutically effective amount of a formulation provided herein.

In one embodiment, provided herein are methods for treating, preventing or ameliorating a nitrogen mustard skin injury in a subject, wherein the methods comprise contacting the subject with a therapeutically effective amount of a formulation provided herein.

In one embodiment, the methods provided herein comprise contacting a subject with a formulation provided herein prior to, during, or after exposure to sulfur mustard. In certain embodiments, the methods provided herein comprise contacting a subject at risk for exposure to sulfur mustard.

In one embodiment, the methods provided herein comprise contacting a subject with a formulation provided herein following an exposure of the subject to sulfur mustard. In certain embodiments, the subject is contacted with a formulation provided herein immediately after the exposure of the subject to sulfur mustard. In one embodiment, the subject is contacted with a formulation provided herein within about 1 minute to about 24 hours after the exposure of the subject to sulfur mustard. In one embodiment, the subject is contacted with a formulation provided herein within about 1 minute to about 1 hour after the exposure of the subject to sulfur mustard. In one embodiment, the subject is contacted with a formulation provided herein within about 1 minute to about 15 minutes after the exposure of the subject to sulfur mustard. In one embodiment, the subject is contacted with a formulation provided herein within about 1 minute to about 5 minutes after the exposure of the subject to sulfur mustard. In one embodiment, the subject is contacted with the formulation provided herein at the onset of symptoms of skin injury due to the exposure to sulfur mustard. In certain embodiments, the subject is contacted with a formulation provided herein for at least 15 minutes after exposure to sulfur mustard. In certain embodiments, the subject is contacted with a formulation provided herein for at least 30 minutes after exposure to sulfur mustard. In certain embodiments, the subject is contacted with a formulation provided herein for at least an hour after exposure to sulfur mustard. In certain embodiments, the subject is contacted with a formulation provided herein for at least a day after exposure to sulfur mustard. In certain embodiments, the subject is contacted with a formulation provided herein for at least a week after exposure to sulfur mustard.

In one embodiment, the methods provided herein comprise contacting a subject with a formulation provided herein following an exposure of the subject to sulfur mustard, wherein the contacting is repeated more than once. In one embodiment, the methods provided herein comprise contacting a subject with a formulation provided herein following an exposure of the subject to sulfur mustard, wherein the contacting is repeated 1 to 10 times. In one embodiment, the methods provided herein comprise contacting a subject with a formulation provided herein following an exposure of the subject to sulfur mustard, wherein the contacting is repeated for 1, 2, 3, 4, 5, 6, 7, 8, 9, or 10 times.

In one embodiment, the methods provided herein comprise contacting a subject with a formulation provided herein following an exposure of the subject to sulfur mustard, wherein the contacting is for at least 1 half-life of sulfur mustard. In one embodiment, the methods provided herein comprise contacting a subject with a formulation provided herein following an exposure of the subject to sulfur mustard, wherein the contacting is for 1 to 10 half-lives of sulfur mustard. In one embodiment, the methods provided herein comprise contacting a subject with a formulation provided herein following an exposure of the subject to sulfur mustard, wherein the contacting is for 1 to 8 half-lives of sulfur mustard. In one embodiment, the methods provided herein comprise contacting a subject with a formulation provided herein following an exposure of the subject to sulfur mustard, wherein the contacting is for 1 to 5 half-lives of sulfur mustard. In one embodiment, the methods provided herein comprise contacting a subject with a formulation provided herein following an exposure of the subject to sulfur mustard, wherein the contacting is for 1 to 3 half-lives of sulfur mustard. In one embodiment, the methods provided herein comprise contacting a subject with a formulation provided herein following an exposure of the subject to sulfur mustard, wherein the contacting is for 1, 2, 3, 4, 5, 6, 7, 8, 9 or 10 half-lives of sulfur mustard.

In one embodiment, the methods provided herein comprise contacting a subject at least once with a unit dosage formulation provided herein following an exposure of the subject to sulfur mustard. In one embodiment, the methods provided herein comprise contacting a subject one to ten times with a unit dosage formulation provided herein following an exposure of the subject to sulfur mustard. In one embodiment, the methods provided herein comprise contacting a subject once, twice, three times, four times, five times, six times, seven times, eight times, nine times or ten times with a unit dosage formulation provided herein following an exposure of the subject to sulfur mustard.

In certain embodiments, the subject is contacted with a formulation provided herein immediately after the exposure of the subject to sulfur mustard. In one embodiment, the subject is contacted with a formulation provided herein within about 1 minute to about 24 hours after the exposure of the subject to sulfur mustard. In one embodiment, the subject is contacted with a formulation provided herein within about 1 minute to about 1 hour after the exposure of the subject to sulfur mustard. In one embodiment, the subject is contacted with a formulation provided herein within about 1 minute to about 15 minutes after the exposure of the subject to sulfur mustard. In one embodiment, the subject is contacted with a formulation provided herein within about 1 minute to about 5 minutes after the exposure of the subject to sulfur mustard. In one embodiment, the subject is contacted with the formulation provided herein at the onset of symptoms of skin injury due to the exposure to sulfur mustard. In certain embodiments, the subject is contacted with a formulation provided herein for at least 15 minutes exposure to sulfur mustard. In certain embodiments, the subject is contacted with a formulation provided herein for at least 30 minutes exposure to sulfur mustard. In certain embodiments, the subject is contacted with a formulation provided herein for at least an hour after exposure to sulfur mustard. In certain embodiments, the subject is contacted with a formulation provided herein for at least a day after exposure to sulfur mustard. In certain embodiments, the subject is contacted with a formulation provided herein for at least a week.

In one embodiment, the formulation provided herein is applied topically and directly to the area exposed to or expected to get exposed to sulfur mustard. In one embodiment, the formulation provided herein is applied directly onto a dressing pad or other appropriate dressing material, and the pad or dressing material is then placed over the area exposed to or expected to get exposed to sulfur mustard.

In a further embodiment, the skin or surface exposed to sulfur mustard is soaked in the formulation by dispensing a pressurized form of the formulation onto the skin or the surface, or applying a coating or layer of the formulation on the skin or surface.

In one embodiment, provided herein is a method comprising contacting the skin, wound or surface that has been exposed to sulfur mustard, with an amount of the formulation that is sufficient to decontaminate the skin or surface. Actual dosage levels of active ingredients in the formulation may be varied so as to obtain an amount of the active compound that is effective to achieve the desired therapeutic response for a particular subject and mode of administration. It is within the skill of the art to determine the dosage required to achieve the desired therapeutic effect.

In one embodiment, the subject is a mammal. In another embodiment, the subject is a human.

In certain embodiments, provided herein are methods of decontaminating a surface exposed to sulfur mustard comprising contacting the surface with a formulation provided herein.

In certain embodiments, provided herein are methods of decontaminating a surface exposed to nitrogen mustard comprising contacting the surface with a formulation provided herein.

### Combination Therapy

In some embodiments, the formulation provided herein may also be combined or used in combination with other therapeutic agents useful in the treatment and/or prevention of conditions associated with exposure to sulfur mustard.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a subject with a disease or disorder. A first therapy (e.g., a prophylactic or therapeutic agent such as a compound provided herein) can be administered prior to (e.g., about 5 minutes, about 15 minutes, about 30 minutes, about 45 minutes, about 1 hour, about 2 hours, about 4 hours, about 6 hours, about 12 hours, about 24 hours, about 48 hours, about 72 hours, about 96 hours, about 1 week, about 2 weeks, about 3 weeks, about 4 weeks, about 5 weeks, about 6 weeks, about 8 weeks, or about 12 weeks before), concomitantly with, or subsequent to (e.g., about 5 minutes, about 15 minutes, about 30 minutes, about 45 minutes, about 1 hour, about 2 hours, about 4 hours, about 6 hours, about 12 hours, about 24 hours, about 48 hours, about 72 hours, about 96 hours, about 1 week, about 2 weeks, about 3 weeks, about 4 weeks, about 5 weeks, about 6 weeks, about 8 weeks, or about 12 weeks after) the administration of a second therapy (e.g., a prophylactic or therapeutic agent) to the subject. Triple therapy is also contemplated herein.

As used herein, the term “synergistic” includes a combination of the formulation provided herein and another therapy (e.g., a prophylactic or therapeutic agent) which has been or is currently being used to treat, prevent, or manage a disease or disorder, which is more effective than the additive effects of the therapies. A synergistic effect of a combination of therapies (e.g., a combination of prophylactic or therapeutic agents) permits the use of lower dosages of one or more of the therapies and/or less frequent administration of said therapies to a subject with a disorder. The ability to utilize lower dosages of a therapy (e.g., a prophylactic or therapeutic agent) and/or to administer said therapy less frequently reduces the toxicity associated with the administration of said therapy to a subject without reducing the efficacy of said therapy in the prevention or treatment of a disorder). In addition, a synergistic effect can result in improved efficacy of agents in the prevention or treatment of a disorder. Finally, a synergistic effect of a combination of therapies (e.g., a combination of prophylactic or therapeutic agents) may avoid or reduce adverse or unwanted side effects associated with the use of either therapy alone.

The formulations provided herein can be administered in combination or alternation with another therapeutic agent. In combination therapy, effective dosages of two or more agents are administered together, whereas in alternation or sequential-step therapy, an effective dosage of each agent is administered serially or sequentially. The dosages given will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions.

The formulation provided herein can be administered in combination with other agents useful for surface decontamination after chemical or biological warfare agent exposure. In one embodiment, the formulation provided herein can be administered in combination with Reactive Skin Decontamination Lotion (RSDL). In certain embodiments, RSDL comprises Dekon 139 and 2,3-butanedione monoxime (DAM) (e.g., with Dekon 139 and DAM being dissolved in a solvent composed of polyethylene glycol monomethyl ether (MPEG) and water). In certain embodiments, the RSDL is impregnated in a sponge pad packaged in a kit as a single unit in a heat-sealed foil pouch. In another embodiment, the formulation provided herein is included with the RSDL kit. Additionally, exemplary agents useful for surface decontamination after chemical or biological warfare agent exposure are disclosed in U.S. Patent Publication Nos. 2022/0023174, 2009/0048153 and 2004/0235756, and U.S. Pat. No. 8,952,023.

In certain embodiments, the formulations provided herein can be administered in combination with other classes of compounds, including, but not limited to, steroids, such as prednisone and dexamethasone; and anti-histamines such as loratadine, cetirizine, diphenhydramine and fexofenadine.

The disclosure will be further understood by the following non-limiting examples.

### EXAMPLES

As used herein, the symbols and conventions used in these experiments, processes, schemes and examples, regardless of whether a particular abbreviation is specifically defined, are consistent with those used in the contemporary scientific literature, for example, the Journal of the American Chemical Society or the Journal of Biological Chemistry. Specifically, but without limitation, the following abbreviations may be used in the examples and throughout the specification:
ACN—acetonitrile
CMC—carboxymethyl cellulose
DCM—dichloromethane
DMSO—dimethyl sulfoxide
eq. or equiv.—equivalent
g—grams
HD—sulfur mustard
HPLC—high pressure liquid chromatography
hr or hrs—hours
JHA—Job Hazard Assessment
k—reaction rate
mL—milliliters
μL—microliters
mM—millimolar
μM—micromolar
L—liters
M—molar
mg—milligrams
min—minutes
mPa·s—Millipascal-second
NMR—nuclear magnetic resonance
nmol—nanomolar
RT—room temperature
s—seconds
SDS—sodium dodecyl sulfate
SODIUM THIOSULFATE—sodium thiosulfate
t_{1/2}—half-life

For all of the following experiments and the following example, standard work-up and purification methods known to those skilled in the art can be utilized. Unless otherwise indicated, all temperatures are expressed in ° C. (degrees Centigrade). All reactions were conducted at room temperature unless otherwise noted. Methodologies illustrated in the following examples are intended to exemplify the applicable chemistry through the use of specific examples and are not indicative of the scope of the invention.

Proton NMR spectra were measured at 300 mHz on a Varian Inova 300 spectrometer. ¹H chemical shifts were referenced to residual Acetone-d5 at 2.05 ppm or DMSO-d₆ at 2.50 ppm. Data was processed and overlaid with iNMR reader. High pressure liquid chromatography analyses were performed using an Agilent series 1100 HPLC instrument with a Grace Alltima column C18 5, 250×4.6 mm, flow: 1 mL/min at 40° C. Elution was isocratic using mixtures of water, A1 (made up of 700 mL water, 300 mL MeOH, 3 mL Et3N, and enough phosphoric acid to give a pH of 3.4) and MeOH. Reactions were monitored at 215 nm.

Sulfur mustard is cytotoxic and a severe blister agent. A Job Hazard Assessment (JHA) was performed before initiation of all activities. Appropriate safety measures were identified during the JHA and were implemented during the synthesis and handling of sulfur mustard. All areas of potential contamination were wiped down with a 0.1 M Oxone in 85:15 water/N-methyl-2-pyrrolidone solution.

### Experiment 1: Sulfur Mustard Reaction with Water and Acetonitrile

Sulfur mustard is a viscous oil having density of 1.237 g/mL. Sulfur mustard is virtually insoluble in water. The hydrolysis rate measurement studies for sulfur mustard were conducted in a mixed solvent system 2.25:1 acetonitrile:water.

TABLE 1

| | | | | | Vol | | | Conc |
|---|---|---|---|---|---|---|---|---|
| | Mol. Wt. | Density | Concentration | Mg | in μL | mEquiv | Rel. eq. | (M) |
| Sulfur | 159.08 | 1.237 | — | 3.711 | 3.0 | 0.02333 | 1.00 | 0.0359 |
| mustard | | | | | | | | |
| Water | 18.02 | 1 | — | 200 | 200 | 11.09878 | 475.8 | 17.08 |
| acetonitrile | | | | | 450 | | | |

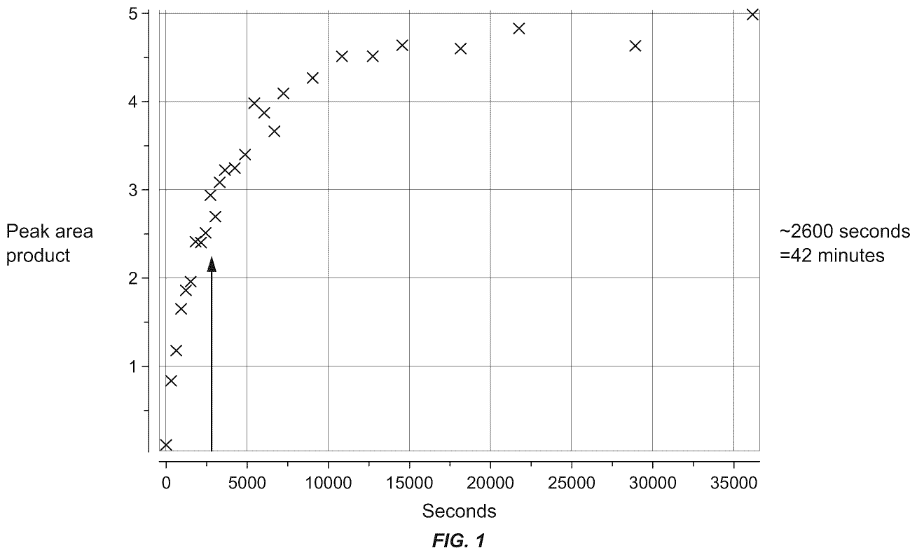

As demonstrated in FIG. 1, sulfur mustard has a hydrolytic half-life of about 42 minutes in a mixed solvent system 2.25:1 acetonitrile:water.

### Example 2: Sulfur Mustard Reaction with Commercially Available Sodium Thiosulfate Injection Solution

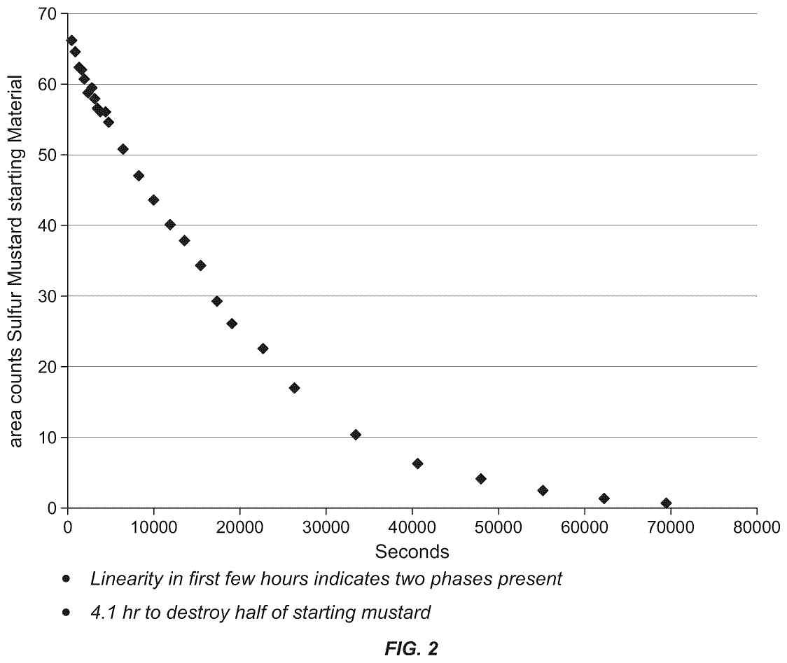

FIG. 2 provides overall kinetics of the reaction between sulfur mustard in contact with commercially available, FDA-approved Sodium Thiosulfate Injection which is an aqueous solution that contains 250 milligrams of sodium thiosulfate per milliliter of solution. The half-life of sulfur mustard with sodium thiosulfate in this two-phase system was 4.1 hours.

NMR experiments revealed the following reaction scheme (Scheme 1) with limited evidence for the formation of a sulfonium-activated intermediate species during the reaction between sulfur mustard and sodium thiosulfate.

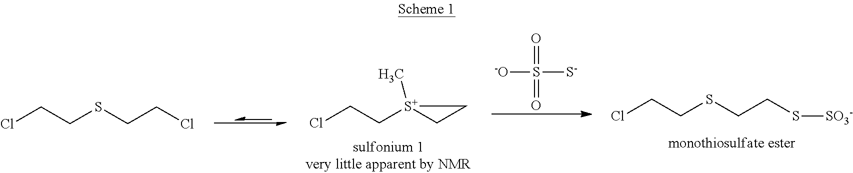

### Experiment 3: Sulfur Mustard Reaction with Sodium Thiosulfate in Different Solvents

Experiments were conducted to find conditions that allowed the reaction of sulfur mustard with sodium thiosulfate to proceed more rapidly than either the sulfur mustard:aqueous sodium thiosulfate or the sulfur mustard:water:acetonitrile models.

Sulfur mustard used in this study was prepared using methods known in the literature.

The reaction of sulfur mustard with sodium thiosulfate was studied in different solvent systems. This study was conducted in 4 phases as described below:

### Phase 1: Reaction of Sodium Octyl Thiosulfate and Sulfur Mustard

Since sulfur mustard is not soluble in D₂O and sodium thiosulfate is not soluble in common organic solvents that could be used to dissolve sulfur mustard, sodium octyl thiosulfate and sodium hexadecyl thiosulfate were designed as model compounds that would mimic sodium thiosulfate but dissolve in the same organic solvents that would dissolve sulfur mustard (Scheme 2).

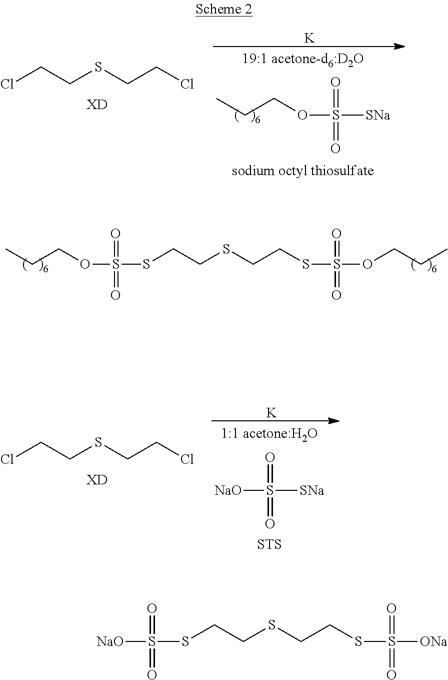

The reaction of sulfur mustard with sodium octyl thiosulfate or sodium thiosulfate:

Solubility trials were conducted to find a solvent system that would dissolve both the sodium thiosulfate model compound (sodium octyl or hexadecyl thiosulfate) and sulfur mustard. The goal was to identify a solvent in which both compounds are soluble enough to give concentrations easily observable by ¹H NMR and retain the sodium thiosulfate/sulfur mustard ratio of the nitrogen mustard experiment. The following deuterated solvents (methanol-d₄, acetone-d₆, DMSO-d₆, acetonitrile-d₃, DMF-d₇, and D₂O) were tested. It was determined that the best model system was sodium octyl thiosulfate as the sodium thiosulfate model compound and 19:1 acetone-d₆:D₂O as the solvent system.

A stock solution of sodium octyl thiosulfate was prepared by dissolving the compound (52 mg) in 19:1 acetone-d₆:D₂O (1.3 mL) with gentle heating. This gave a slightly turbid solution (40 mg/mL, 0.1611 mM).

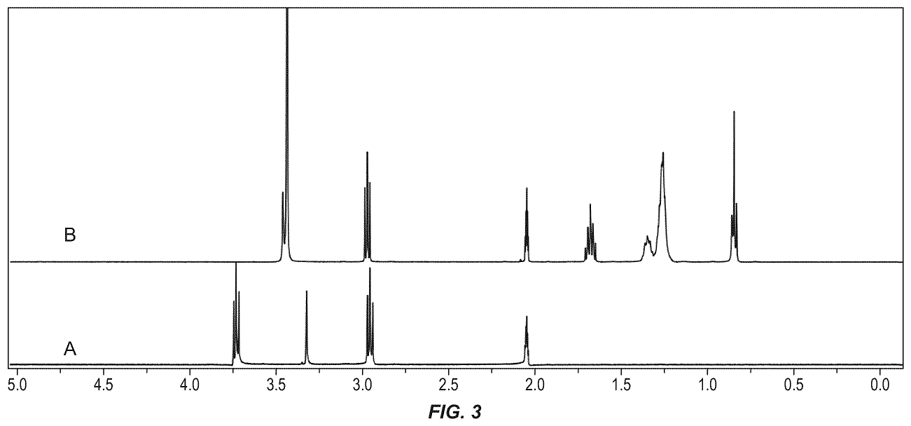

A stock solution of sulfur mustard was prepared by dissolving sulfur mustard (23 mg) in 19:1 acetone-d₆:D₂O (2.7 mL) to give a stock solution of sulfur mustard (8.52 mg/mL, 0.0535 mM). The ¹H NMR spectrum of each stock solution is provided in FIG. 3, where A is ¹H NMR spectrum for sulfur mustard) and B is ¹H NMR spectrum for sodium octyl thiosulfate.

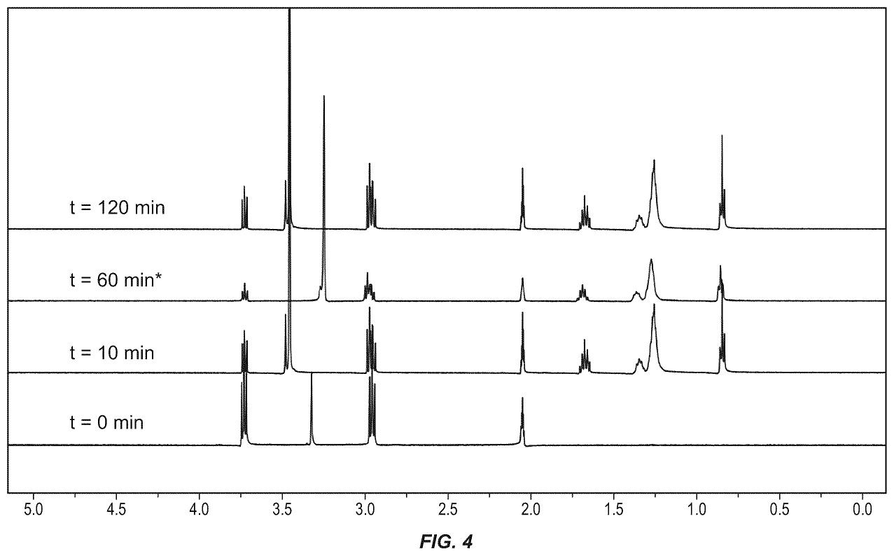

The sulfur mustard stock solution (0.5 mL) was placed in an NMR tube and the spectrum collected (t=0 min). Then the sodium octyl thiosulfate stock solution (1 mL) was added, and the instrument parameters (shimming) were adjusted. Spectra were collected at 10, 15, 20, 25, 30, 35, 40, 45, 50, and 55 min. Then the temperature was increased from 25° C. to 45° C. and spectra were collected at 60, 70, 80, 90, 100, 110, and 120 min. As seen from the ¹H NMR spectra provided in FIG. 4, no reaction was observed.

### Phase 2: Reactions Between Sulfur Mustard and Sodium Thiosulfate in Mixtures of Water and Organic Solvents:

The results of the Phase 1 ¹H NMR experiment as well as observations in the literature showed that the rate of reaction of sulfur mustard with nucleophiles such as thiosulfate(s) is governed by the polarity of the solvent, see Scheme 3 below and Wang Q-Q., Begum R. A., Day V. W., Bowman-James K., Sulfur, oxygen, and nitrogen mustards: stability and reactivity, Org. Biomol. Chem., 2012, 10, 8786; Yang Y-C., Ward J. R., Luteran T., Hydrolysis of Mustard Derivatives in Aqueous Acetone-Water and Ethanol-Water Mixtures, J. O. C., 1996 51(14) 2756-2759; and Yang Y-C., Szafraniec L. L., Beaudry W. T., Ward J. R., Kinetics and Mechanism of the Hydrolysis of 2-Chloroethyl Sulfides, J. O. C., 1988 53(14) 3293-3297.

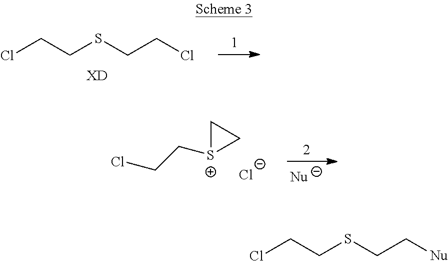

Reaction of sulfur mustard with nucleophiles. 1. Unimolecular formation of the reactive intermediate is dependent upon the polarity of the solvent. 2. Reaction of the cyclic sulfonium intermediate with nucleophiles

In relatively non-polar 19:1 acetone:water, the reaction of sulfur mustard with octyl thiosulfate was sluggish to the point that product could not be observed after two hours and at elevated temperatures.

The Phase 1 solubility studies showed that sulfur mustard and sodium thiosulfate could be dissolved in some of the same mixtures of water and organic solvents. This eliminated the need to use sodium octyl sulfate as a model compound. A higher proportion of water was thought to increase the polarity of the reaction mixture, and so increase the rate of reaction between sulfur mustard and sodium thiosulfate. Phase 1 also showed that ¹H NMR was an extremely effective, but cumbersome method of monitoring the course of sulfur mustard/sodium thiosulfate reactions. To allow for higher throughput, an HPLC method for monitoring the reaction was devised.

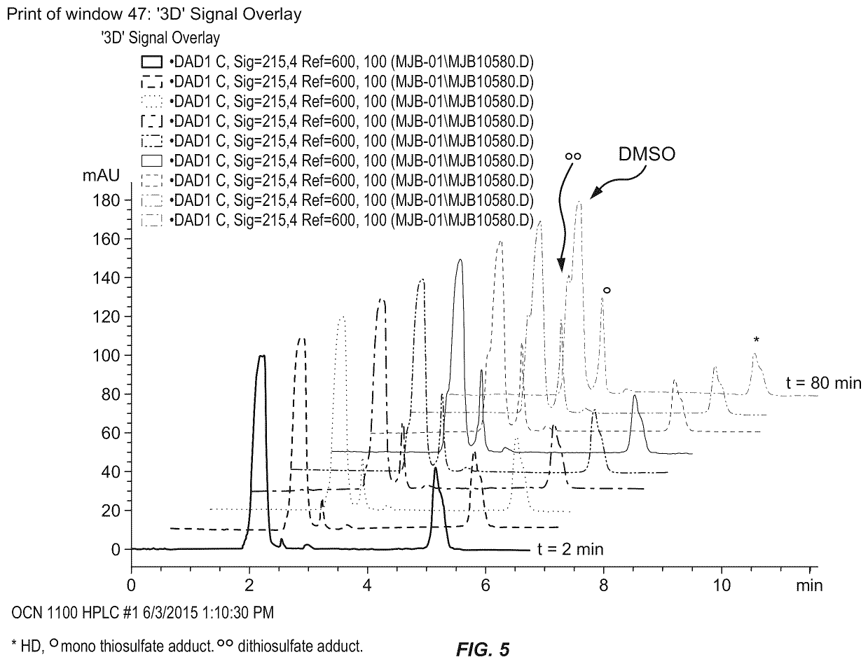

To test the effect of solvent polarity on reaction rate, sulfur mustard was mixed with a homogeneous solution of 1:1 acetone:water containing 200 mM sodium thiosulfate. This gave a suspension of sulfur mustard (20 mg/mL). Sulfur mustard was maintained in suspension with periodic shaking. A 1 μL aliquot of this suspension was analyzed by HPLC at 2, 10, 20, 30, 40, 50, 60, 70, and 80 min. Under these conditions the half-life of sulfur mustard was 70-80 min, determined by the total peak area of sulfur mustard in the HPLC trace (FIG. 5). This result showed that increasing the proportion of water in the reaction mixture increases the reaction rate (when compared to the ¹H NMR experiment), that sodium thiosulfate is very soluble in water/organic mixtures and that HPLC was a fast and effective method of monitoring this reaction. Because sulfur mustard was only partially soluble in 1:1 acetone:water, the experiment also made it clear that the solubility of sulfur mustard in water/organic mixtures was the limiting factor.

To address this problem, the limit of solubility of sulfur mustard in different water organic mixtures was determined. The solubility is concentration dependent, and it was decided to aim for a 10 mg/mL solution for ease of further experiments. Sulfur mustard was dissolved in the organic solvent and water was added to give the desired water/organic ratio and sulfur mustard concentration. The mixture was shaken for 10 sec and sulfur mustard was deemed soluble if a clear solution was obtained. A cloudy or turbid suspension indicated insolubility. Sulfur mustard was freely soluble in 30% water/DMSO, 30% water/MeOH, 50% water/ACN, 50% water/DMF, and 50% water/acetone at this concentration. Complimentarily, sodium thiosulfate was soluble at 94 mg/mL (6 eq/mL to the sulfur mustard stock solution) in all of these water/organic mixtures except the acetonitrile water mixture where the proportion of water had to be adjusted to 70% or a biphasic solution would form.

The rate of reaction in each water/organic mixture was tested with an end-point HPLC assay. In the experiment, 0.5 mL of a 10 mg/mL sulfur mustard solution (in each organic/water mixture) was mixed with 0.5 mL of the 94 mg/mL sodium thiosulfate solution (in the corresponding water/organic mixture—6 eq). The extent of the reaction was monitored after one hour by comparing the total peak area of the sulfur mustard after 60 min with the 0 min time point (Table 2).

TABLE 2
The reaction of sulfur mustard (10 mg/mL) with sodium
thiosulfate (94 mg/mL) in different solvent systems

| | Remaining sulfur mustard |
|---|---|
| Water/Organic Mixture | after 60 min (%) |
| 30% water/DMSO | 47 |
| 50% water/CAN | 26 |
| 50% water/DMF | 19 |
| 50% water/acetone | 49 |
| 30% water/MeOH | 54 |

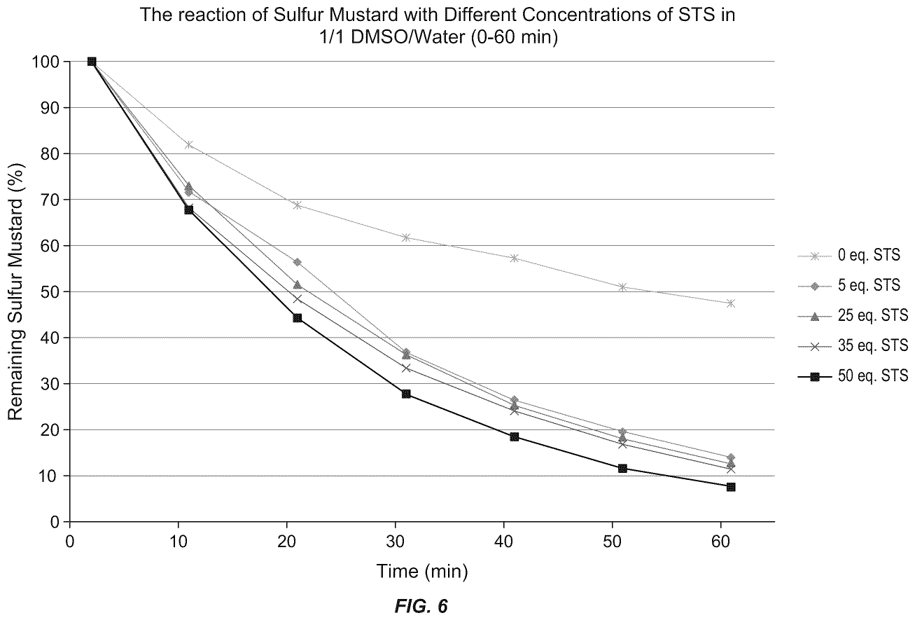

Of the organic solvents tested, DMSO was the solvent of choice for future experiments because it is the most favorable for clinical or field applications. In order to further increase the observed rate of reaction between sulfur mustard and sodium thiosulfate, the concentration of sulfur mustard in the reactions was decreased to 5 mg/mL so that a higher proportion of water could be used in the reaction mixture. At this concentration sulfur mustard, was freely soluble in 50% water/DMSO. The rate of degradation of sulfur mustard in this solvent system was then assayed with different concentrations of sodium thiosulfate (FIG. 6). All concentrations of sodium thiosulfate used gave a similar rate of reaction (between 18 and 25 minutes half-life). This is in agreement with the literature that specifies that sulfur mustard breaks down via a unimolecular mechanism (Scheme 3). These results demonstrated that the rate of degradation is not dependent upon the concentration of the second reactant.

Phase 3: 1H NMR Analysis of the Reaction of Sulfur Mustard with Sodium Thiosulfate in 1/1 DMSO-d₆/D₂O:

The Phase 2 experiments furnished a solvent system in which both sulfur mustard and sodium thiosulfate were soluble at concentrations sufficient to analyze the reaction. The experiments also provided a new HPLC method to analyze the reaction. Analysis with HPLC showed a half-life of approximately 20 minutes with 10-50 equivalents of sodium thiosulfate to sulfur mustard (FIG. 6). The following experiments were conducted to analyze the rate of the reaction under these conditions with ¹H NMR.

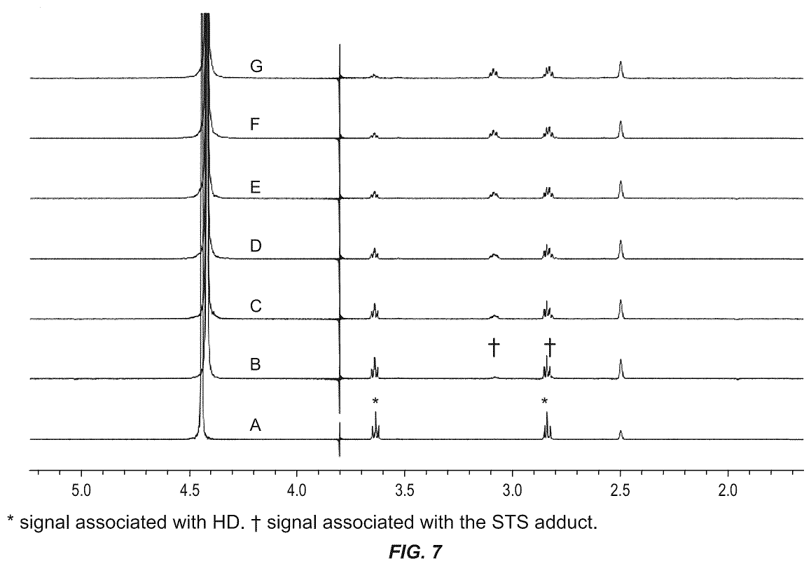

For the ¹H NMR experiment a 5 mg/mL stock solution of sulfur mustard and a 78 mg/mL stock solution of sodium thiosulfate (10 eq.) were prepared in 1/1 DMSO-d₆/D₂O. The sulfur mustard stock was made as close to experiment time as possible to minimize hydrolysis in the aqueous solution. The solutions were mixed in equal proportions and the ¹H NMR spectrum was acquired as soon as instrument parameters could be adjusted (1-2 min). Additional spectra were acquired every 10 min for 100 min (FIG. 7).

The ratio of sulfur mustard starting material to sodium thiosulfate adduct was approximated from the integration (size) ratio of the signal at 3.6 ppm and the signal at 3.2 ppm. The signal at 3.6 ppm corresponds to the hydrogens on the methylene carbon in sulfur mustard that is bound to chlorine and the signal at 3.2 ppm corresponds to the hydrogens on the methylene carbon of the sodium thiosulfate adduct that is bound to sulfur (Scheme 4).

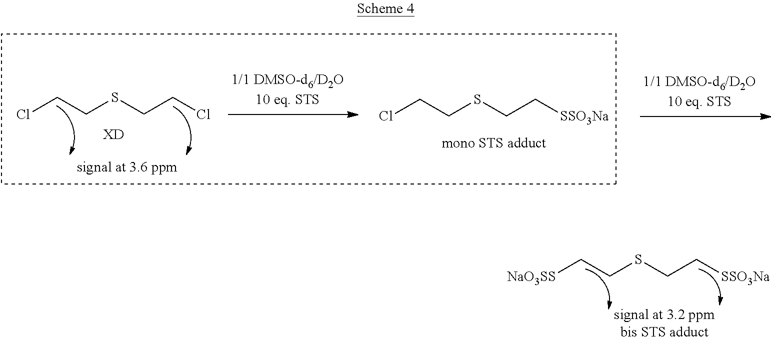

The reaction of sulfur mustard with sodium thiosulfate, formation of mono- and bis-sodium thiosulfate adducts. Boxed reaction is simplified reaction used to find the half-life with ¹H NMR.

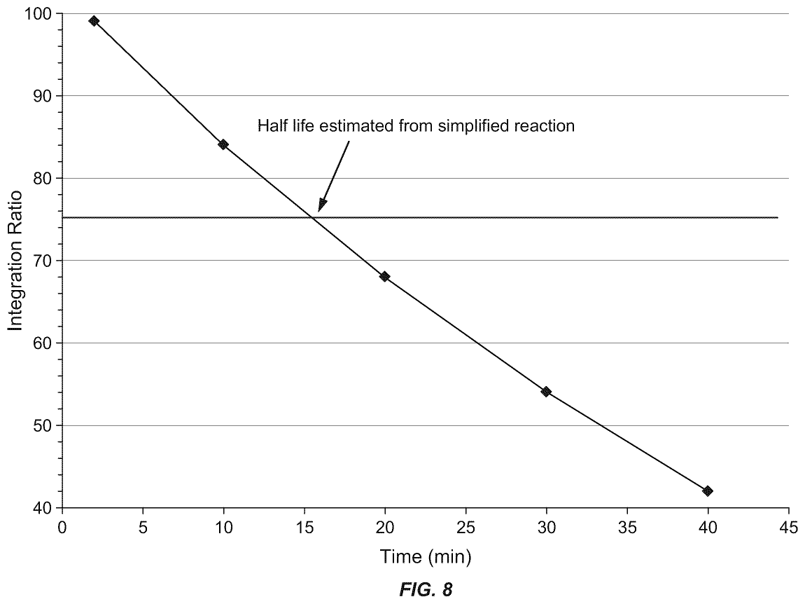

Sulfur mustard has two reactive sites, forming mono- and bis-sodium thiosulfate adducts. These three species are not resolved in ¹H NMR. Only the protons on the two different types of carbon (chloride bound or sulfur bound) are separated. So, the integration ratio of the 3.2 and 3.6 ppm signals corresponds to the number of methylenes that are sulfur or chloride bound, not the ratio of the three different molecular species (sulfur mustard, mono-, bis-sodium thiosulfate adducts). However, the amount of starting sulfur mustard can be estimated from the simplified reaction for the formation of the mono-sodium thiosulfate adduct only (boxed reaction, scheme 4). In the simplified reaction, the integration ratio was expected to be (3.6 ppm/(3.2+3.6 ppm) when half of the sulfur mustard is consumed to be 75% (FIG. 8) and to be 50% when all sulfur mustard is consumed. The simplified reaction was used to generate a half-life of approximately 16 minutes (FIG. 8).

### Phase 4: Additions to DMSO/Water Mixtures:

Having determined that the half-life of the reaction of sulfur mustard with sodium thiosulfate in 1/1/DMSO water is approximately 20 minutes by HPLC and approximately 16 minutes by ¹H NMR, additional parameters were sought to obtain a short enough half-life to make the reaction clinically useful. The first experiments tried were with ACN/water mixtures because the Phase 2 end point assay indicated that the reaction might proceed faster in this solvent system. However, more thorough investigation showed that the reaction proceeded at a similar rate in DMSO/water and ACN/water mixtures. The effect of pH was then tested. The pH of an unbuffered sodium thiosulfate solution is around 4. When pH 7 (sodium phosphate) or pH 10 (sodium carbonate/bicarbonate) buffers were used in place of pure water there was not a significant effect on the reaction rate. Other salts that might increase the ionic strength of the solution or drive the reaction to completion by precipitation of chloride ions were tested. Calcium carbonate and ammonium sulfate did not positively influence the rate of reaction. Silver nitrate, silver citrate, and lead acetate did not mix well with sodium thiosulfate and did not have a positive effect on the reaction rate.

Next a series of soaps and emulsifiers were tested to see if a higher proportion of water could be used and increase the reaction rate (Table 3).

TABLE 3
Solubility of Sulfur Mustard (5 mg/mL) in
aq. soap/DMSO mixtures (%) to dissolve

| | Proportion aqueous |
|---|---|
| | (%) to dissolve |
| | Sulfur Mustard |
| Soap/Emulsifier | (5 mg/mL) |
| 10% PEG 1500 | 60 |
| 2% alfa-cyclodextrin | 60 |
| 2% tetrabutylammonium bromide | 50 |
| 1% sodium dodecyl sulfate | 60 |
| 2% sodium dodecyl sulfate | 80 |
| 4% sodium dodecyl sulfate | 90 |
| 10% triton X-100 | 100 |
| 5% triton X-100 | 95 |
| 3% triton X-100 | 90 |
| 5% cetyltrimethylammonium carbonate | 95 |
| 2% cetyltrimethylammonium carbonate | 90 |
| 1% cetyltrimethylammonium carbonate | 70 |

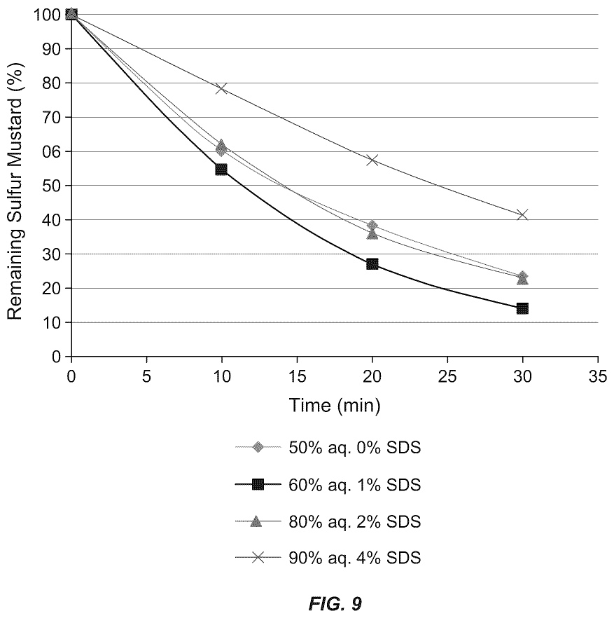

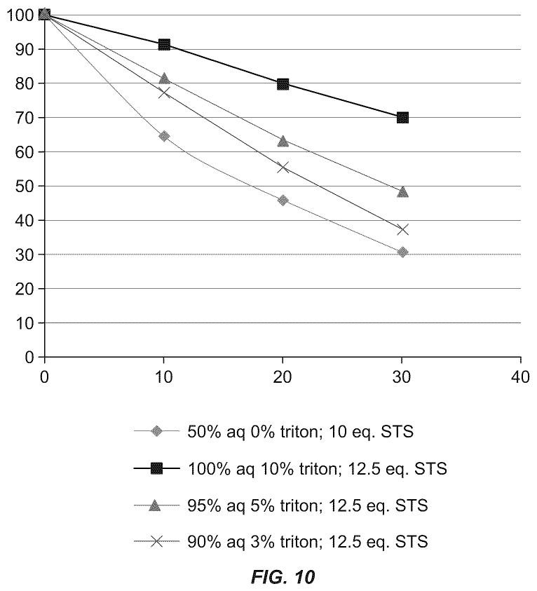

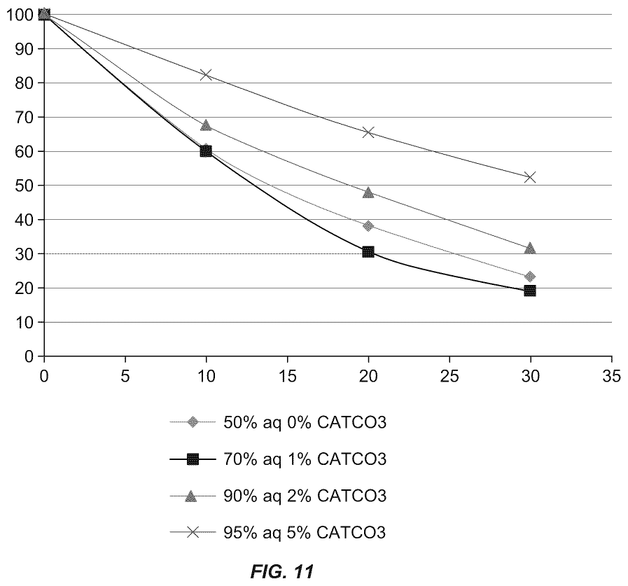

In all proportions, triton X-100 slowed the reaction rate, even with higher concentrations of water. The other soaps showed small increases in reaction rates at low concentrations, but the reaction rate slowed with higher soap concentrations even though more water was used in the reactions (FIGURES. 9-11).

### Conclusions

Several water/organic solvent mixtures were found that dissolve significant quantities of both sodium thiosulfate and sulfur mustard. These solvent systems along with a fast and effective HPLC method and more traditional ¹H NMR experiments allowed the study of the rate of reaction between sodium thiosulfate and sulfur mustard. It was found that sodium thiosulfate degrades sulfur mustard with a half-life of approximately 18 min (50 eq. sodium thiosulfate) in 1/1 DMSO/water when assayed with HPLC. A slightly shorter half-life of approximately 16 min (10 eq. sodium thiosulfate) was observed with ¹H NMR for the same solvent system. By trying a variety of conditions, it was found that this reaction rate is not heavily influenced by the pH of the solution, the presence of other salt ions, or the concentration of sodium thiosulfate above 6 equivalents. Based on the results herein, the factor influencing the rate of the reaction is the polarity of the solvent medium. The reaction rate could be increased slightly by adding small amounts of a soap/emulsifier to increase the proportion of water in the reaction, but larger amounts slowed the reaction rate. The systems that gave the fastest observed reaction times with a half-life of 13 min for 80% aq. 2% sodium docedyl sulfate (SDS)/DMSO (50 eq. sodium thiosulfate) found by HPLC.

### Experiment 4: Sulfur Mustard Reaction with Sodium Thiosulfate in Aqueous Solutions with Different Modifiers

The reaction rates and reaction half-lives for the decomposition of sulfur mustard in aqueous solutions using different modifiers was investigated and compared to the reaction rates and reaction half-lives for the decomposition of sulfur mustard with sodium thiosulfate added to those solutions. A timed degradation study was used to determine the reaction rate and reaction half-life of sulfur mustard in various aqueous solutions.

Unless otherwise noted, reactions were performed without exclusion of air or moisture. All reagents were purchased from TCI, Oakwood, or Sigma-Aldrich, and sulfur mustard (prepared as described below) was used for all timed degradation studies.

### Analytical Methods

Analytical thin layer chromatography (TLC) was performed using aluminum plates precoated with 0.25 mm of 230-400 mesh silica gel impregnated with a fluorescent indicator (254 nmol). TLC plates were visualized by exposure to KMnO₄ stain. NMR spectra were recorded on a Bruker Avance III HD 500 equipped with a prodigy multinuclear cryoprobe (1H: 500 MHz) NMR spectrometer at the University of Oregon NMR facilities. 1H chemical shifts (6) are expressed in ppm relative to the residual CHCl₃ (1H: 7.26 ppm). For simplicity, the JH-H coupling constants of protons in all 1H spectra have been reported as pseudo first-order when possible. Individual spectra can be identified by their notebook number.

High pressure liquid chromatography analyses were performed using an ° ° ° ent series 1100 HPLC instrument with a Grace Alltima column and method HOP_G1 (Table 4). Chromatograms were manually integrated. Individual chromatograms are assigned an automatic HPLC identification number.

TABLE 4
HPLC Method HOP_G1
Method Conditions for Purity by HPLC

| Instrument | Agilent series 1100 HPLC equipped with | | |
|---|---|---|---|
| | DAD Detector and ChemStation | | |
| Column | Grace Alltima C18, 250 × 4.6 mm, 5 μm, | | |
| | Part Number 88056 | | |
| Column Temperature | 40° C. | | |
| Mobile Phase A (MPA) | 0.05% Trifluoroacetic acid in Water | | |
| Mobile Phase B (MPB) | 0.05% Trifluoroacetic acid in Acetonitrile | | |
| Diluent/Needlewash | Acetonitrile | | |
| Injection Volume | 10 μL | | |
| Detection Wavelength | 215 nm | | |
| Flow Rate | 1.5 mL/min | | |
| Standard diluent | Experiment Specific | | |
| | Time (Min) | % MPA | % MPB |
| HPLC Gradient | 0.00 | 50 | 50 |
| Parameters | 5.00 | 5 | 95 |
| | 6.00 | 5 | 95 |
| | 6.10 | 50 | 50 |
| | 8.00 | 50 | 50 |

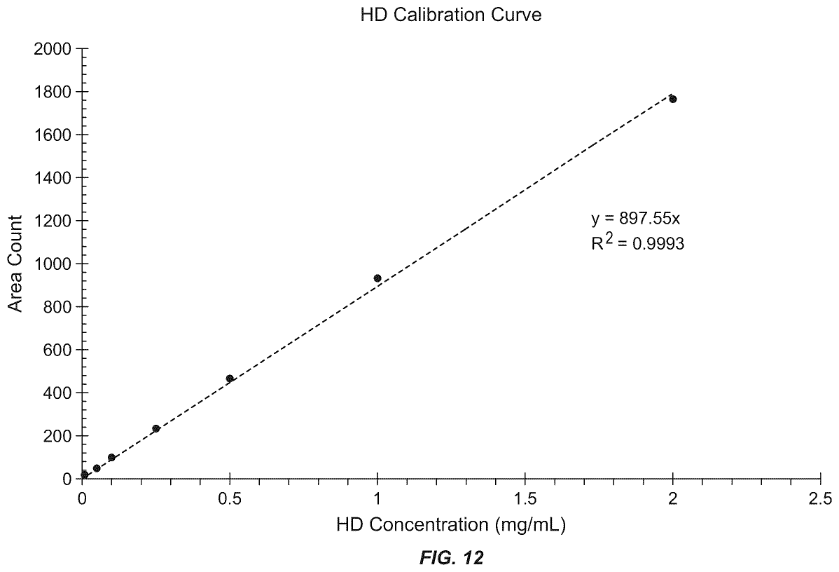

To determine concentrations of sulfur mustard in HPLC samples, a 7-point calibration curve spanning 0.01-2.00 mg/mL was prepared by HPLC analysis of 0.10, 0.25, 0.50, 1.00, and 2.00 mg/mL solutions of sulfur mustard in acetonitrile (see solution preparation section below). For data points for 0.05 and 0.01 mg/mL sulfur mustard concentrations, HPLC injection volume was set at 5 and 1 μL of the 0.10 mg/mL sulfur mustard solution respectively. A standard calibration curve based on the area of the peak at each dilution was then created. The calibration curve can be seen in FIG. 12. Concentrations were calculated using the slope of the best fit line (y=897.55x) where x=concentration (in mg/mL) and y=area of the HPLC signal.

All pH Measurements were made with a Hanna Instruments HI 4222 pH meter and a Hanna instruments HI 1330B micro pH probe or a Hanna instruments HI 1131B pH probe. The pH meter was calibrated before each use and the results documented in the pH calibration logbook.

### Synthesis of Sulfur Mustard

Sulfur mustard used in this study was prepared using methods known in the literature. The sulfur mustard is distilled with an apparent boiling point of 126-129° C. to furnish sulfur mustard as a clear oil (5.29 g, 81.4% yield). 1H NMR (500 MHz, CDCl₃) δ 3.69-3.61 (m, 2H), 2.97-2.88 (m, 2H); HPLC (method HOP_G1): tR=5.32 min, 99.9% purity.

### Solution Preparations

For all solution preparations, accurate dilutions were made using the appropriate 10, 50, 200 or 1000 μL gas tight Hamilton syringe, or appropriately sized class A volumetric glassware. For solid reagents, samples were weighed using a Mettler Toledo XS105 analytical balance (accurate to +/−0.01 mg).

### Sulfur Mustard Solutions

100 mg/mL Sulfur Mustard Standard Solution: A 3 mL glass vial was charged with 1.00 mL of HPLC grade Acetonitrile (Fisher item #A998-4, lot #216640). Sulfur mustard (HOP-01-02, 78.7 μL, 100 mg) was added to the vial and thoroughly mixed. This stock solution of sulfur mustard was used throughout the course of all experiments. To ensure there was no drift in the concentration over time, each day a timed degradation study was initiated, a fresh 0.50 or 1.00 mg/mL HPLC check standard was prepared and the concentration was verified against the HPLC calibration curve (see FIG. 12). Over the course of this project, the check standard did not show variation outside of experimental error.

2.00 mg/mL Sulfur Mustard in Acetonitrile: An HPLC vial was changed with 1.00 mL of HPLC grade Acetonitrile (Fisher item #A998-4, lot #216640) and 20.0 μL of the 100 mg/mL sulfur mustard standard solution was added. The vial was tightly capped and thoroughly mixed before use.

1.00 mg/mL Sulfur Mustard in Acetonitrile: An HPLC vial was changed with 1.00 mL of HPLC grade Acetonitrile (Fisher item #A998-4, lot #216640) and 10.0 μL of the 100 mg/mL sulfur mustard standard solution was added. The vial was tightly capped and thoroughly mixed before use.

0.50 mg/mL Sulfur Mustard in Acetonitrile: An HPLC vial was changed with 1.00 mL of HPLC grade Acetonitrile (Fisher item #A998-4, lot #216640) and 5.0 μL of the 100 mg/mL sulfur mustard standard solution was added. The vial was tightly capped and thoroughly mixed before use.

0.25 mg/mL Sulfur Mustard in Acetonitrile: An HPLC vial was changed with 1.00 mL of HPLC grade Acetonitrile (Fisher item #A998-4, lot #216640) and 2.5 μL of the 100 mg/mL sulfur mustard standard solution was added. The vial was tightly capped and thoroughly mixed before use.

0.10 mg/mL Sulfur Mustard in Acetonitrile: An HPLC vial was changed with 1.00 mL of HPLC grade Acetonitrile (Fisher item #A998-4, lot #216640) and 1.0 μL of the 100 mg/mL sulfur mustard standard solution was added. The vial was tightly capped and thoroughly mixed before use.

### Emulsification Solutions in Water

1% n-Decyl Sodium Sulfate: n-Decyl sodium sulfate (Oakwood item #001075, lot #001075P20X, 106.48 mg) was weighed in to a clean, tarred 20 mL scintillation vial. To the vial, 10.0 mL of HPLC water (Fisher item #W5-4, lot #217919) was added via volumetric pipette and the vial was mixed until all solid material had visibly dissolved.

1% Sodium Lauryl Sulfate: Sodium lauryl sulfate (Oakwood item #098465, lot #098465I20D, 100.51 mg) was weighed in to a clean, tarred 20 mL scintillation vial. To the vial, 10.0 mL of HPLC water (Fisher item #W5-4, lot #217919) was added via volumetric pipette and the vial was mixed until all solid material had visibly dissolved.

1% Triethanolamine: Triethanolamine (Oakwood item #242387, lot #242387J16K, 99.94 mg) was weighed in to a clean, tarred 20 mL scintillation vial. To the vial, 10.0 mL of HPLC water (Fisher item #W5-4, lot #217919) was added via volumetric pipette and the vial was mixed until all solid material had visibly dissolved.

1% Tetrabutylammonium Chloride: Tetrabutylammonium chloride (Oakwood item #053205, lot #053205P19F, 109.77 mg) was weighed in to a clean, tarred 20 mL scintillation vial. To the vial, 10.0 mL of HPLC water (Fisher item #W5-4, lot #217919) was added via volumetric pipette and the vial was mixed until all solid material had visibly dissolved.

1% Methyl Cellulose: Methyl cellulose (Sigma-Aldrich item #M0512-100G, lot #SLCJ8769, 100 mg+10%) was weighed in to a clean, tarred 20 mL scintillation vial. To the vial, 10.0 mL of HPLC water (Fisher item #W5-4, lot #217919) was added via volumetric pipette and the vial was mixed until all solid material had visibly dissolved. Mild heating, sonication and vortexing was required for complete dissolution of methyl cellulose.

1% Carboxymethyl Cellulose Sodium Salt: Carboxymethyl cellulose sodium salt (Sigma-Aldrich item #C5678-500G, lot #SLCK1957, 100 mg+10%) was weighed in to a clean, tarred 20 mL scintillation vial. To the vial, 10.0 mL of HPLC water (Fisher item #W5-4, lot #217919) was added via volumetric pipette and the vial was mixed until all solid material had visibly dissolved. Mild heating, sonication and vortexing was required for complete dissolution of carboxymethyl cellulose sodium salt.

Emulsification Solutions with 10% Sodium Thiosulfate

10% Sodium Thiosulfate in Water (HOP-01-15): Sodium thiosulfate pentahydrate (Fisher item #S445-500, lot #210331, 10.008 g) was weighed in to a clean, tarred 100 mL volumetric flask and approximately 90 mL of HPLC water (Fisher item #W5-4, lot #217919) was added and the flask was mixed until all solid material had dissolved. The flask was then diluted to 100.0 mL with additional HPLC water and mixed.

1% n-Decyl Sodium Sulfate, 10% Sodium Thiosulfate: n-Decyl sodium sulfate (Oakwood item #001075, lot #001075P20X, 101.79 mg) was weighed in to a clean, tarred 20 mL scintillation vial. To the vial, 10.0 mL of 10% sodium thiosulfate in water (HOP-O1-15) was added via volumetric pipette and the vial was mixed until all solid material had visibly dissolved.

1% Sodium Lauryl Sulfate, 10% Sodium Thiosulfate: Sodium lauryl sulfate (Oakwood item #098465, lot #098465I20D, 100.35 mg) was weighed in to a clean, tarred 20 mL scintillation vial. To the vial, 10.0 mL of 10% sodium thiosulfate in water (HOP-O1-15) was added via volumetric pipette and the vial was mixed until all solid material had visibly dissolved.

1% Triethanolamine, 10% Sodium Thiosulfate: Triethanolamine (Oakwood item #242387, lot #242387J16K, 100.51 mg) was weighed in to a clean, tarred 20 mL scintillation vial. To the vial, 10.0 mL of 10% sodium thiosulfate in water (HOP-O1-15) was added via volumetric pipette and the vial was mixed until all solid material had visibly dissolved.

1% Methyl Cellulose, 10% Sodium Thiosulfate: Methyl cellulose (Sigma-Aldrich item #M0512-100G, lot #SLCJ8769, 100.94 mg) was weighed in to a clean, tarred 20 mL scintillation vial. To the vial, 10.0 mL of 10% sodium thiosulfate in water (HOP-O1-15) was added via volumetric pipette and the vial was mixed. Despite extensive mixing, sonication and heating, insoluble material remained in the vial. This solution was not used for timed degradation studies.

1% Carboxymethyl Cellulose Sodium Salt, 10% Sodium Thiosulfate: Carboxymethyl cellulose sodium salt (Sigma-Aldrich item #C5678-500G, lot #SLCK1957, 102.66 mg) was weighed in to a clean, tarred 20 mL scintillation vial. To the vial, 10.0 mL of 10% sodium thiosulfate in water (HOP-01-15) was added via volumetric pipette and the vial was mixed until all solid material had visibly dissolved. Mild heating, sonication and vortexing was required for complete dissolution of carboxymethyl cellulose sodium salt.

### 50 mM Borate Buffer pH 8.5 (HOP-01-23)

Boric acid (Mallinkrodt item #2549, lot #2549N16617, 3.09 g) was weighed into a 1.0 L media bottle with a stir bar and 800 mL of HPLC water (Fisher item #W5-4, lot #217919) was added. The solution was stirred until all the boric acid had dissolved and the pH was adjusted to 8.5 with sodium hydroxide (Fisher item #S318-3, lot #172340). The solution was then diluted with HPLC water to a final volume of 1.0 L. The final pH was measured as 8.53.

Emulsification Solutions with 50 mM Borate Buffer

1% n-Decyl Sodium Sulfate: n-Decyl sodium sulfate (Oakwood item #001075, lot #001075P20X, 100.39 mg) was weighed in to a clean, tarred 20 mL scintillation vial. To the vial, 10.0 mL of 50 mM borate buffer pH 8.5 (HOP-01-23) was added via volumetric pipette and the vial was mixed until all solid material had visibly dissolved. The pH of this solution was measured as 8.51.

1% Sodium Lauryl Sulfate: Sodium lauryl sulfate (Oakwood item #098465, lot #098465I20D, 104.47 mg) was weighed in to a clean, tarred 20 mL scintillation vial. To the vial, 10.0 mL of 50 mM borate buffer pH 8.5 (HOP-01-23) was added via volumetric pipette and the vial was mixed until all solid material had visibly dissolved. The pH of this solution was measured as 8.54.

1% Triethanolamine: Triethanolamine (Oakwood item #242387, lot #242387J16K, 101.64 mg) was weighed in to a clean, tarred 20 mL scintillation vial. To the vial, 10.0 mL of 50 mM borate buffer pH 8.5 (HOP-01-23) was added via volumetric pipette and the vial was mixed until all solid material had visibly dissolved.

1% Methyl Cellulose: Methyl cellulose 80-120 mPa·s, 2% in water at 20° C. (TCI item #M0292, lot #08HRG-DN, 105.40) was weighed in to a clean, tarred 20 mL scintillation vial. To the vial, 10.0 mL of 50 mM borate buffer pH 8.5 (HOP-01-23) was added via volumetric pipette and the vial was mixed until all solid material had visibly dissolved. Mild heating, sonication and vortexing was required for complete dissolution of methyl cellulose. The pH of this solution was measured as 8.45.

1% Carboxymethyl Cellulose Sodium Salt: Carboxymethyl cellulose sodium salt (Sigma-Aldrich item #C5678-500G, lot #SLCK1957, 102.08 mg) was weighed in to a clean, tarred 20 mL scintillation vial. To the vial, 10.0 mL of 50 mM borate buffer pH 8.5 (HOP-01-23) was added via volumetric pipette and the vial was mixed until all solid material had visibly dissolved. Mild heating, sonication and vortexing was required for complete dissolution of carboxymethyl cellulose sodium salt. The pH of this solution was measured as 8.53.

Emulsification Solutions with Sodium Thiosulfate and 50 mM Borate Buffer

10% Sodium Thiosulfate in 50 mM Borate Buffer pH 8.5 (HOP-01-30): Sodium thiosulfate pentahydrate (Fisher item #S445-500, lot #210331, 10.044 g) was weighed in to a clean, tarred 100 mL volumetric flask and approximately 90 mL of 50 mM borate buffer pH 8.5 (HOP-O1-15) was added and the flask was mixed until all solid material had dissolved. The flask was then diluted to 100.0 mL with an additional 50 mM borate buffer pH 8.5 and mixed.

5% Sodium Thiosulfate in 50 mM Borate Buffer pH 8.5 (HOP-01-35_1): Sodium thiosulfate pentahydrate (Fisher item #S445-500, lot #210331, 5.010 g) was weighed in to a clean, tarred 100 mL volumetric flask and approximately 90 mL of 50 mM borate buffer pH 8.5 (HOP-O1-15) was added and the flask was mixed until all solid material had dissolved. The flask was then diluted to 100.0 mL with an additional 50 mM borate buffer pH 8.5 and mixed.

1% Sodium Thiosulfate in 50 mM Borate Buffer pH 8.5 (HOP-01-35_2): Sodium thiosulfate pentahydrate (Fisher item #S445-500, lot #210331, 1.001 g) was weighed in to a clean, tarred 100 mL volumetric flask and approximately 90 mL of 50 mM borate buffer pH 8.5 (HOP-01-15) was added and the flask was mixed until all solid material had dissolved. The flask was then diluted to 100.0 mL with an additional 50 mM borate buffer pH 8.5 and mixed.

1% n-Decyl Sodium Sulfate, 10% Sodium Thiosulfate: n-Decyl sodium sulfate (Oakwood item #001075, lot #001075P20X, 102.26 mg) was weighed in to a clean, tarred 20 mL scintillation vial. To the vial, 10.0 mL of 10% sodium thiosulfate in 50 mM borate buffer pH 8.5 (HOP-01-30) was added via volumetric pipette and the vial was mixed until all solid material had visibly dissolved. The pH of this solution was measured as 8.22.

1% Sodium Lauryl Sulfate, 10% Sodium Thiosulfate: Sodium lauryl sulfate (Oakwood item #098465, lot #098465I20D, 105.11 mg) was weighed in to a clean, tarred 20 mL scintillation vial. To the vial, 10.0 mL of 10% sodium thiosulfate in 50 mM borate buffer pH 8.5 (HOP-01-30) was added via volumetric pipette and the vial was mixed until all solid material had visibly dissolved. The pH of this solution was measured as 8.22.

1% Sodium Lauryl Sulfate, 5% Sodium Thiosulfate: Sodium lauryl sulfate (Oakwood item #098465, lot #098465I20D, 104.68 mg) was weighed in to a clean, tarred 20 mL scintillation vial. To the vial, 10.0 mL of 5% sodium thiosulfate in 50 mM borate buffer pH 8.5 (HOP-01-35_1) was added via volumetric pipette and the vial was mixed until all solid material had visibly dissolved.

1% Sodium Lauryl Sulfate, 1% Sodium Thiosulfate: Sodium lauryl sulfate (Oakwood item #098465, lot #098465I20D, 102.67 mg) was weighed in to a clean, tarred 20 mL scintillation vial. To the vial, 10.0 mL of 1% sodium thiosulfate in 50 mM borate buffer pH 8.5 (HOP-01-35_2) was added via volumetric pipette and the vial was mixed until all solid material had visibly dissolved.

1% Methyl Cellulose, 10% Sodium Thiosulfate: Methyl cellulose (Sigma-Aldrich item #M0512-100G, lot #SLCJ8769, 105.24 mg) was weighed in to a clean, tarred 20 mL scintillation vial. To the vial, 10.0 mL of 10% sodium thiosulfate in 50 mM borate buffer pH 8.5 (HOP-01-30) was added via volumetric pipette and the vial was mixed. Despite extensive mixing, sonication and heating, insoluble material remained in the vial. This solution was not used for timed degradation studies.

1% Methyl Cellulose, 1% Sodium Thiosulfate: Methyl cellulose (Sigma-Aldrich item #M0512-100G, lot #SLCJ8769, 101.34 mg) was weighed in to a clean, tarred 20 mL scintillation vial. To the vial, 10.0 mL of 1% sodium thiosulfate in 50 mM borate buffer pH 8.5 (HOP-01-35_2) was added via volumetric pipette and the vial was mixed. Mild heating, sonication and vortexing was required for complete dissolution of methyl cellulose.

1% Carboxymethyl Cellulose Sodium Salt, 10% Sodium Thiosulfate: Carboxymethyl cellulose sodium salt (Sigma-Aldrich item #C5678-500G, lot #SLCK1957, 102.66 mg) was weighed in to a clean, tarred 20 mL scintillation vial. To the vial, 10.0 mL of 10% sodium thiosulfate in 50 mM borate buffer pH 8.5 (HOP-01-30) was added via volumetric pipette and the vial was mixed until all solid material had visibly dissolved. Mild heating, sonication and vortexing was required for complete dissolution of carboxymethyl cellulose sodium salt. The pH of this solution was measured as 8.21.

### Timed Degradation Study

HPLC vials were allotted with 1.00 mL of reaction solution. A solution of 5 or 10 μL of a 100 mg/mL sulfur mustard stock solution in acetonitrile was diluted to 1.00 mL with acetonitrile, analyzed, and the value was assumed to be equal to the zero-time point. For the degradation time points, 5 or 10 μL of the 100 mg/mL sulfur mustard stock solution was injected into the HPLC vials containing 1.00 mL of reaction solution (target concentration of 0.5 or 1.0 mg/mL) and the vial was mixed on a vortex mixer for 30 seconds. The HPLC vial was immediately transferred to an HPLC for analysis and sampled every 10 minutes from 2.5 to 122.5 or 2.5 to 62.5 minutes depending on the specific experiment. The reaction half-life was determined via Equation 1 where t %/2 is the reaction half-life and k is the rate constant.

$${t}_{1 / 2} = \frac{ln 2}{k} \qquad Equation 1$$

The reaction rate k was determined by plotting −ln[sulfur mustard] vs time and determining the slope of the best fit line. The concentration of sulfur mustard ([HD]) in the HPLC vials was determined using a standard calibration curve (see FIG. 12). Data points after 90% decomposition of sulfur mustard (based on initial starting concentration) were excluded. Experiments were run at room temperature with no temperature control (temperatures ranged from 19 to 25° C. for individual experiments). For the experiments using unbuffered water, a single timed degradation study was conducted. For the experiments using 50 mM borate buffered water (pH=8.5) experiments were run in triplicate and the averaged results were used to determine reaction rates and reaction half-lives. No external reference standard was used in this study, and it was assumed based on experimental and literature precedence that the decomposition of sulfur mustard can be approximated using 1st order kinetics.

The reaction rates and reaction half-lives were determined for aqueous solutions containing 1% modifier and 1% modifier with 10% sodium thiosulfate and 50 mM borate buffered (pH 8.5) aqueous solutions containing 1% modifier and 1% modifier with 1-10% sodium thiosulfate. The following modifiers were used:

n-Decyl Sodium Sulfate

The decomposition of sulfur mustard was examined in four solutions containing n-decyl sodium sulfate as the modifier. The individual solutions studied were 1% n-decyl sodium sulfate in unbuffered water, 1% n-decyl sodium sulfate in 50 mM borate buffer, 1% n-decyl sodium sulfate, 10% sodium thiosulfate in unbuffered water, and 1% n-decyl sodium sulfate, 10% sodium thiosulfate in 50 mM borate buffer. FIG. 12 shows the plot of ln[sulfur mustard] vs time for the decomposition of sulfur mustard in these solutions while Table 5 shows the reaction rate k and the calculated half-life of each solution. As can be seen in FIG. 12 and Table 5, the reaction rate approximately doubles upon the addition of sodium thiosulfate to unbuffered water and also doubles when using 50 mM borate buffer vs unbuffered water. The reaction rate is unchanged when sodium thiosulfate is added to the 50 mM borate buffered system. The reaction half-life in unbuffered water was determined to be 29 minutes while the other three solutions tested had half-lives between 11.4 and 14.3 minutes.

TABLE 5
Reaction rates and calculated half-lives for the
decomposition of sulfur mustard in solutions containing
n-decyl sodium sulfate as the modifier.

| | Rate | Reaction |
|---|---|---|
| | Constant | Half-life |
| Reaction Solution | k (s⁻¹) | (min) |
| 1% n-decyl NaSO₄ in unbuffered water | 3.96 × 10⁻⁴ | 29.1 |
| 1% n-decyl NaSO₄ in 50 mM borate buffer | 1.02 × 10⁻³ | 11.4 |
| 1% n-decyl NaSO₄, 10% SODIUM | 9.06 × 10⁻⁴ | 12.7 |
| THIOSULFATE in unbuffered water | | |
| 1% n-decyl NaSO₄, 10% SODIUM | 8.07 × 10⁻⁴ | 14.3 |
| THIOSULFATE in 50 mM borate buffer | | |

### Sodium Lauryl Sulfate

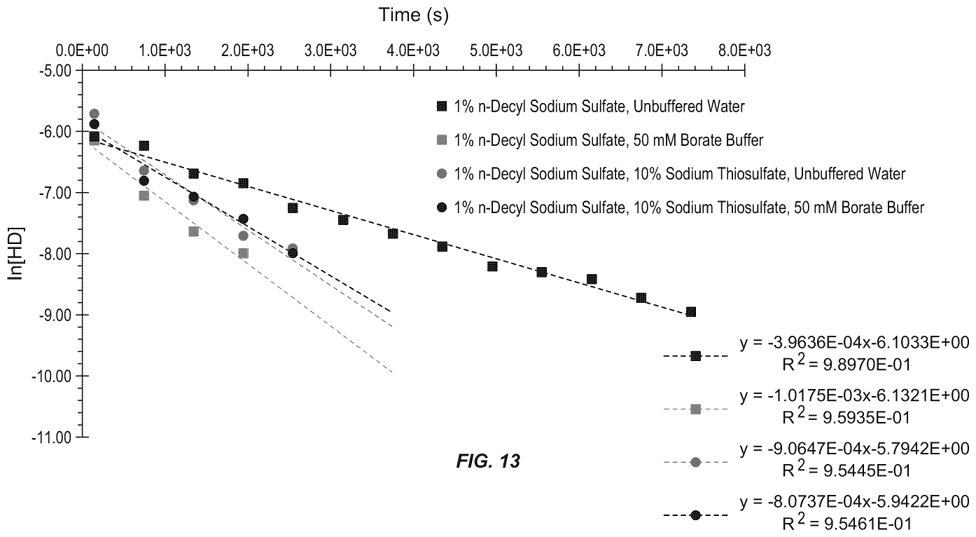

The decomposition of sulfur mustard was examined in six solutions containing sodium lauryl sulfate (n-dodecyl NaSO₄) as the modifier. The individual solutions studied were 1% sodium lauryl sulfate in unbuffered water (solution 1), 1% sodium lauryl sulfate in 50 mM borate buffer (solution 2), 1% sodium lauryl sulfate, 10% sodium thiosulfate in unbuffered water (solution 3), 1% sodium lauryl sulfate, 10% sodium thiosulfate in 50 mM borate buffer (solution 4), 1% sodium lauryl sulfate, 5% sodium thiosulfate in 50 mM borate buffer (solution 5), and 1% sodium lauryl sulfate, 1% sodium thiosulfate in 50 mM borate buffer (solution 6). FIG. 13 shows the plot of ln[sulfur mustard] vs time for the decomposition of sulfur mustard in solutions 1-4 while Table 6 shows the reaction rate k and the calculated half-lives for solutions 1-6. As can be seen in FIG. 13 and Table 6, the reaction rate approximately doubles upon the addition of sodium thiosulfate to either unbuffered water or 50 mM borate buffer. The reaction rate is slightly increased when a 50 mM borate buffer is used vs unbuffered water. The reaction half-life in unbuffered water was determined to be 32 minutes while the half-life in unbuffered water with 10% sodium thiosulfate slightly more than doubled at 12.7 minutes. The borate buffered solutions had half-lives of 20.7 and 7.9 minutes, respectively. Analogous to the unbuffered system, the addition of sodium thiosulfate slightly more than doubled the reaction rate.

TABLE 6
Reaction rates and calculated half-lives for the
decomposition of sulfur mustard in solutions containing
sodium lauryl sulfate as the modifier.

| | Rate | Reaction |
|---|---|---|
| | Constant | Half-life |
| Reaction Solution | k (s⁻¹) | (min) |
| 1% n-dodecyl NaSO₄ in unbuffered water | 3.60 × 10⁻⁴ | 32.1 |
| 1% n-dodecyl NaSO₄ in 50 mM borate buffer | 5.57 × 10⁻⁴ | 20.7 |
| 1% n-dodecyl NaSO₄, 10% SODIUM | 9.06 × 10⁻⁴ | 12.7 |
| THIOSULFATE in unbuffered water | | |
| 1% n-dodecyl NaSO₄, 10% SODIUM | 1.46 × 10⁻³ | 7.9 |
| THIOSULFATE in 50 mM borate buffer | | |
| 1% n-dodecyl NaSO₄, 5% SODIUM | 1.43 × 10⁻³ | 8.1 |
| THIOSULFATE in 50 mM borate buffer | | |
| 1% n-dodecyl NaSO₄, 1% SODIUM | 1.78 × 10⁻³ | 6.5 |
| THIOSULFATE in 50 mM borate buffer | | |

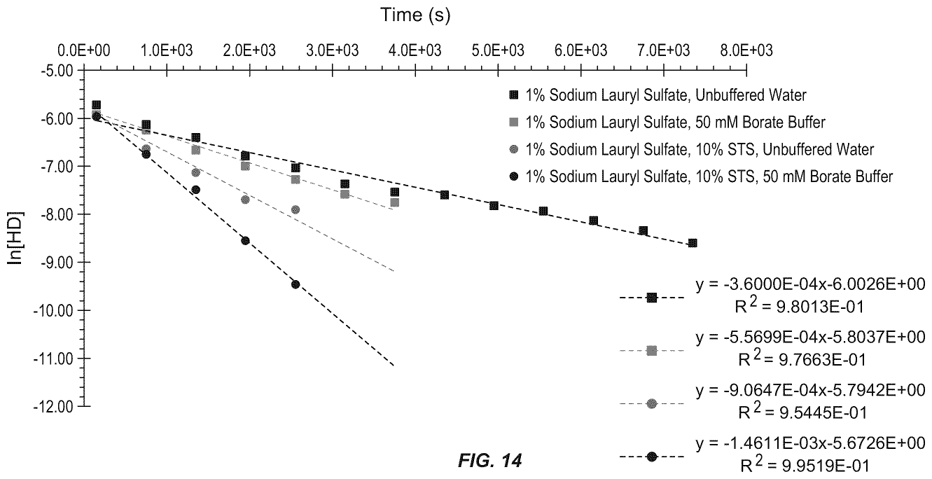

To examine the effect of sodium thiosulfate concentration on the reaction rate, three experiments were run where the concentration of sodium thiosulfate varied from 1-10% (solutions 4-6). The plot of the ln[sulfur mustard] vs time for these experiments is show in FIG. 14 while the reaction rate k and the calculated half-lives are provided in Table 11. As seen in FIG. 14 and Table 11, the reaction rate varies only slightly (within experimental error). This data suggests that the decomposition of sulfur mustard in aqueous solutions follows 1st order rate kinetics and confirms the results in Example 2 that also showed the decomposition of sulfur mustard is independent of the concentration of sodium thiosulfate.

### Triethanolamine

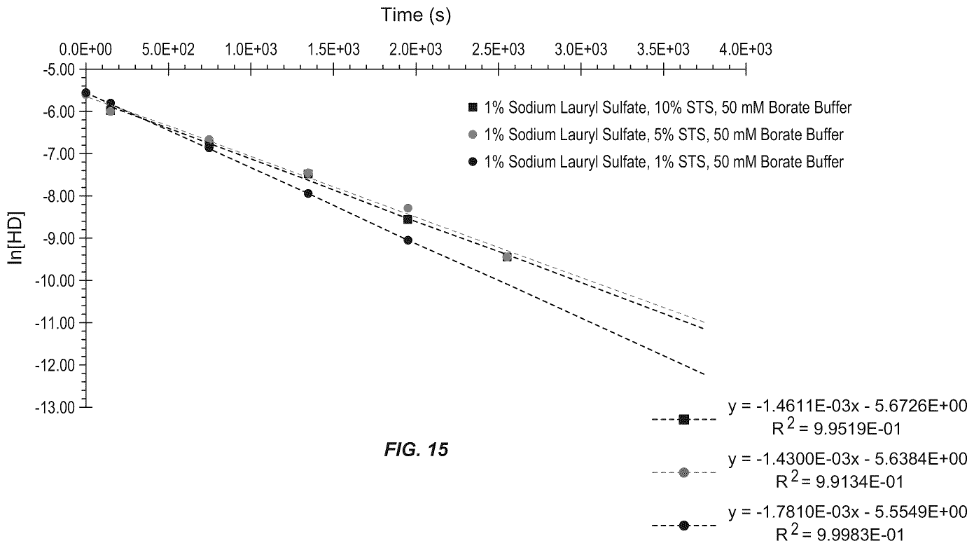

The decomposition of sulfur mustard was examined in three solutions containing triethanolamine as the modifier. The individual solutions studied were 1% triethanolamine in unbuffered water, 1% triethanolamine in 50 mM borate buffer, and 1% triethanolamine, 10% sodium thiosulfate in unbuffered water. FIG. 15 shows the plot of ln[sulfur mustard] vs time for the decomposition of sulfur mustard in these solutions while Table 7 shows the reaction rate k and the calculated half-life of each solution. As can be seen in FIG. 15 and Table 7, the reaction rate approximately doubles upon the addition of sodium thiosulfate to unbuffered water giving half-lives of 30.2 and 13.4 min respectively. Interestingly, moving from unbuffered water to 50 mM borate buffer gave highly variable results. While the calculated reaction rate and half-life in the 50 mM borate buffered solution (3.91×10-4 s-1 and 29.2 min) was similar to the result when using unbuffered water (3.82×10-4 s-1 and 30.2 min), as can be seen in FIG. 15, the R² value for the best fit line for the borate buffered solution was 0.49, significantly worse than the R² values obtained for triethanolamine in unbuffered water (R²=0.95 and 0.99). Based on the high variability observed in the 50 mM borate buffered solution, we elected not to investigate the reaction half-life of sulfur mustard in a 1% triethanolamine, 10% sodium thiosulfate, 50 mM borate buffer solution.

TABLE 7
Reaction rates and calculated half-lives for the decomposition of sulfur
mustard in solutions containing triethanolamine as the modifier.

| | Rate | Reaction |
|---|---|---|
| | Constant | Half-life |
| Reaction Solution | k (s⁻¹) | (min) |
| 1% triethanolamine in unbuffered water | 3.82 × 10⁻⁴ | 30.2 |
| 1% triethanolamine in 50 mM borate buffer | 3.91 × 10⁻³ | 29.6 |
| 1% triethanolamine, 10% SODIUM | 8.28 × 10⁻⁴ | 13.4 |
| THIOSULFATE in unbuffered water | | |

### Tetrabutylammonium Chloride

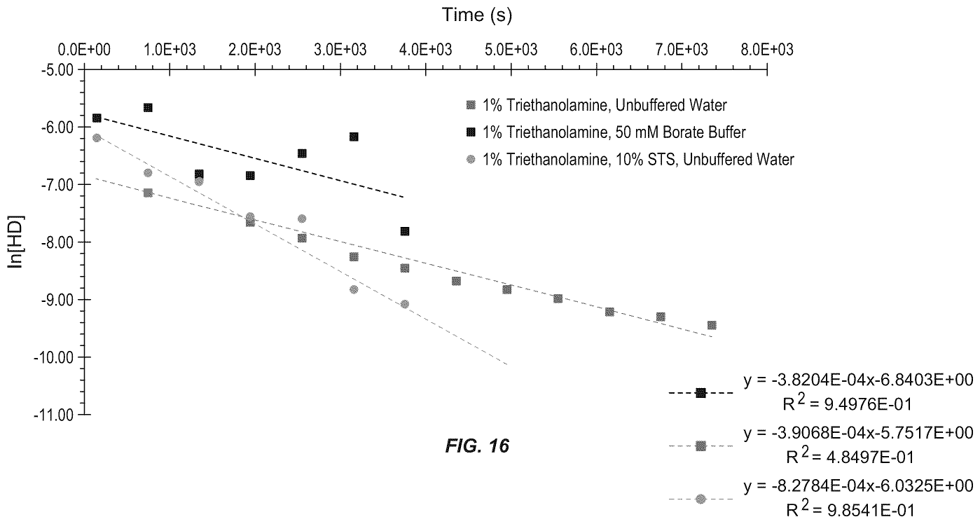

The decomposition of sulfur mustard was examined in one solution containing tetrabutylammonium chloride as the modifier (1% tetrabutylammonium chloride in unbuffered water). FIG. 16 shows the plot of ln[sulfur mustard] vs time for the decomposition of sulfur mustard in this solution while Table 8 shows the reaction rate k and the calculated half-life of this solution. As can be seen in FIG. 16, the plot of the ln[sulfur mustard] vs time does not conform to first order reaction kinetics. Based on this result, we elected to not investigate any further solutions containing tetrabutylammonium chloride as the modifier.

TABLE 8
Reaction rate and calculated half-life for the decomposition
of sulfur mustard in a solution containing tetrabutylammonium
chloride as the modifier.

| | Rate | Reaction |
|---|---|---|
| | Constant | Half-life |
| Reaction Solution | k (s⁻¹) | (min) |
| 1% tetrabutylammonium chloride in | 6.34 × 10⁻⁴ | 18.2 |
| unbuffered water | | |

### Methyl Cellulose

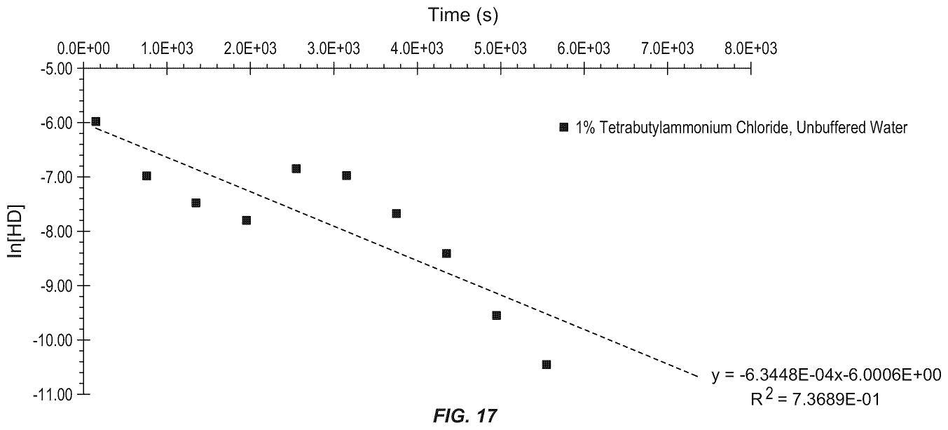

The decomposition of sulfur mustard was examined in three solutions containing methyl cellulose as the modifier. The individual solutions studied were 1% methyl cellulose in unbuffered water, 1% methyl cellulose in 50 mM borate buffer, and 1% methyl cellulose, 1% sodium thiosulfate in 50 mM borate buffer. During formulation of solutions of methyl cellulose with sodium thiosulfate, it was found that at 10% sodium thiosulfate, the mixture was either insoluble or formed a gel which was unable to be analyzed via HPLC. Given the results above that showed that concentrations of sodium thiosulfate ranging from 1-10% gave similar reaction rates and half-life's, it was decided that a solution of 1% methyl cellulose with 1% sodium thiosulfate would yield comparable results. FIG. 17 shows the plot of ln[sulfur mustard] vs time for the decomposition of sulfur mustard in these solutions while Table 9 shows the reaction rate k and the calculated half-life of each solution. As can be seen in FIG. 17 and Table 9, there is no change in the reaction rate between the three conditions tested. The reaction half-life for all three solutions tested was between 7.6 and 8.7 minutes.

TABLE 9
Reaction rates and calculated half-lives for the decomposition of sulfur
mustard in solutions containing methyl cellulose as the modifier.

| | Rate | Reaction |
|---|---|---|
| | Constant | Half-life |
| Reaction Solution | k (s⁻¹) | (min) |
| 1% methyl cellulose in unbuffered water | 1.53 × 10⁻⁴ | 7.6 |
| 1% methyl cellulose in 50 mM borate | 1.33 × 10⁻³ | 8.7 |
| buffer | | |
| 1% methyl cellulose, 1% SODIUM | 1.38 × 10⁻⁴ | 8.4 |
| THIOSULFATE in 50 mM borate buffer | | |

### Carboxymethyl Cellulose

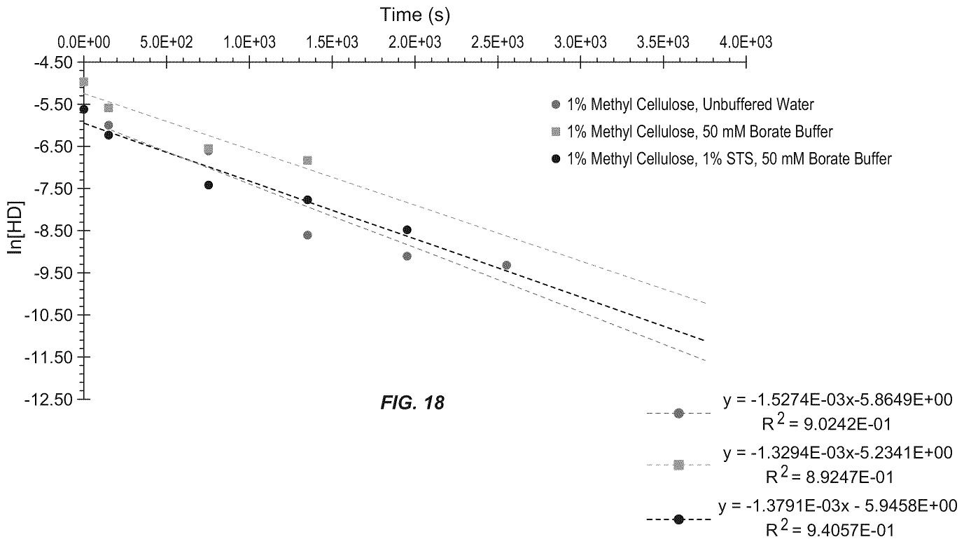

The decomposition of sulfur mustard was examined in four solutions containing carboxy methyl cellulose sodium salt as the modifier. The individual solutions studied were 1% carboxy methyl cellulose sodium salt in unbuffered water, 1% carboxy methyl cellulose sodium salt in 50 mM borate buffer, 1% carboxy methyl cellulose sodium salt, 10% sodium thiosulfate in unbuffered water, and 1% carboxy methyl cellulose sodium salt, 10% sodium thiosulfate in 50 mM borate buffer. FIG. 18 shows the plot of ln[sulfur mustard] vs time for the decomposition of sulfur mustard in these solutions while Table 10 shows the reaction rate k and the calculated half-life of each solution. As can be seen in FIG. 18 and Table 10, the reaction rate is similar for solutions in unbuffered water or 50 mM borate buffer and the reaction rate approximately doubles upon the addition of sodium thiosulfate to both solutions of unbuffered water and 50 mM borate buffer. The reaction half-life in unbuffered water or 50 mM borate buffer was determined to be 10.9 to 13.1 minutes (respectively) while solutions containing 10% sodium thiosulfate had half-lives of 4.9 minutes.

TABLE 10
Reaction rates and calculated half-lives for the decomposition
of sulfur mustard in solutions containing carboxy methyl
cellulose sodium salt as the modifier.

| | Rate | Reaction |
|---|---|---|
| | Constant | Half-life |
| Reaction Solution | k (s⁻¹) | (min) |
| 1% carboxy methyl cellulose in unbuffered | 1.06 × 10⁻³ | 10.9 |
| water | | |
| 1% carboxy methyl cellulose in 50 mM | 8.83 × 10⁻⁴ | 13.1 |
| borate buffer | | |
| 1% carboxy methyl cellulose, | 2.37 × 10⁻³ | 4.9 |
| 10% sodium thiosulfate in unbuffered water | | |
| 1% carboxy methyl cellulose, 10% sodium | 2.37 × 10⁻³ | 4.9 |
| thiosulfate in 50 mM borate buffer | | |

### Results

The reaction rates and reaction half-lives for the decomposition of sulfur mustard in aqueous solutions using different modifiers was investigated and compared to the reaction rates and reaction half-lives for the decomposition of sulfur mustard with sodium thiosulfate added to those solutions. It was found that the decomposition half-life of sulfur mustard varied from 7.6 to 32 minutes in aqueous solutions with different modifiers and that the reaction half-life was correlated with the specific modifier used. The decomposition half-life of sulfur mustard varied from 4.9 to 14 minutes in aqueous solutions with sodium thiosulfate added and that the addition of sodium thiosulfate either had no effect on reaction rate and half-life, or approximately doubled the reaction rate and decreased the reaction half-life by ½. These experiments confirmed that increasing the aqueous content of the reaction solutions would, presumably, increase the reaction rate. The effect of the concentration of sodium thiosulfate on the reaction rate showed no difference in reaction rate or half-life when 10, 5 or 1% sodium thiosulfate solutions were used. These results confirmed that the decomposition of sulfur mustard in aqueous solutions follows 1^{st} order rate kinetics.

The results demonstrated that sulfur mustard could be solubilized in water by modifiers and that the reaction of sulfur mustard with sodium thiosulfate is significantly faster in these mainly aqueous solutions. In this study, it was found that a maximum reaction rate of 2.37×10⁻³ s⁻¹ corresponding to a reaction half-life of 4.9 minutes in a solution of 1% carboxy methyl cellulose sodium salt and 10% sodium thiosulfate was achieved.

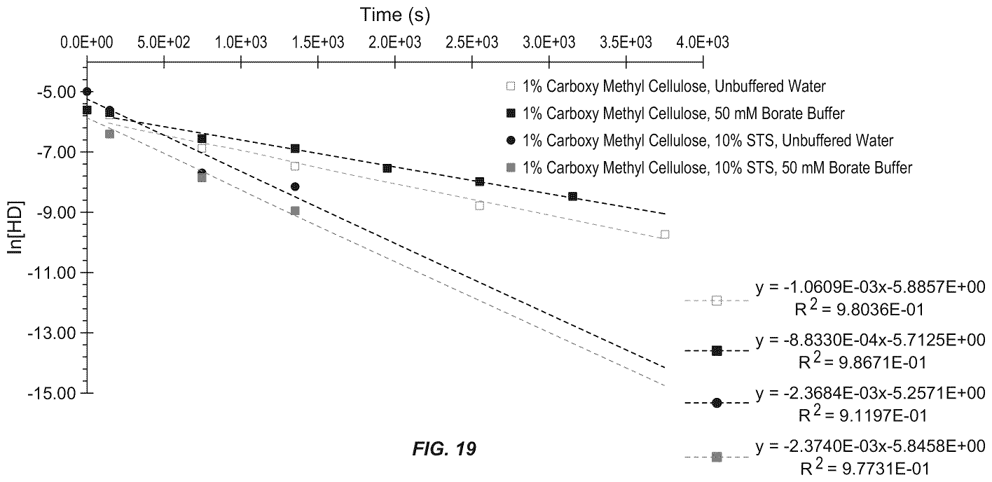

To confirm that the methodology used in this example yielded comparable results to the studies conducted in Example 2, a previous experiment in Example 2 was repeated and the results were compared. The plot of ln[sulfur mustard] vs time can be seen in FIG. 19 for Example 2 methodology and the methodology used in this study. As can be seen in Table 11, the rate constants k and the calculated reaction half-lives between the two studies are in agreement.

TABLE 11
Determined reaction rate constants and reaction half-lives
for conditions in Example 3 compared to Example 2.

| | Rate | Reaction |
|---|---|---|
| | Constant | Half-life |
| Reaction Solvent | k (s⁻¹) | (min) |
| 1:1 DMSO/H₂O (Example 3) | 2.19 × 10⁻⁴ | 52.8 |
| 1:1 DMSO/H₂O (Example 2) | 2.03 × 10⁻⁴ | 57.0 |
| 1:1 DMSO/H₂O, | 4.24 × 10⁻⁴ | 27.2 |
| 12.5% Sodium Thiosulfate | | |
| (Example 3) | | |
| 1:1 DMSO/H₂O, | 5.62 × 10⁻⁴ | 20.6 |
| 5 equiv. Sodium Thiosulfate | | |
| (Example 2) | | |

The examples set forth above are provided to give those of ordinary skill in the art with a complete disclosure and description of how to make and use the claimed embodiments and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

What is claimed is:

1. A formulation comprising sodium thiosulfate, and a cellulose or a pharmaceutically acceptable salt thereof.

2. The formulation of claim 1 further comprising a buffer.

3. The formulation of claim 1, comprising sodium thiosulfate, and carboxymethyl cellulose or a pharmaceutically acceptable salt thereof.

4. The formulation of claim 1, comprising about 0.1% to about 25% sodium thiosulfate based on total weight of the formulation.

5. The formulation of claim 1, comprising about 5% to about 15% sodium thiosulfate based on total weight of the formulation.

6. The formulation of claim 1, comprising about 0.01% to about 5% carboxymethyl cellulose or a pharmaceutically acceptable salt thereof, based on total weight of the formulation.

7. The formulation of claim 2, wherein the buffer is selected from a group consisting of an acetate buffer, a borate buffer, a citrate buffer, a phosphate buffer, and a sulfate buffer.

8. The formulation of claim 7, wherein the buffer is a borate buffer.

9. The formulation of claim 1, comprising about 5% to about 15% sodium thiosulfate, and 0.5% to about 2% carboxymethyl cellulose or a pharmaceutically acceptable salt thereof, based on total weight of the formulation.

10. The formulation of claim 1, comprising about 5% to about 15% sodium thiosulfate, 0.5% to about 2% carboxymethyl cellulose or a pharmaceutically acceptable salt thereof based on total weight of the formulation, and a buffer.

11. The formulation of claim 1, comprising about 10% sodium thiosulfate based on total weight of the formulation.

12. The formulation of claim 1, comprising about 1% carboxymethyl cellulose or a pharmaceutically acceptable salt thereof based on total weight of the formulation.

13. The formulation of claim 1, comprising about 10% sodium thiosulfate, about 1% carboxymethyl cellulose or a pharmaceutically acceptable salt thereof, based on total weight of the formulation, and a 50 mM borate buffer.

14. The formulation of claim 1, comprising carboxymethyl cellulose sodium salt.

15. The formulation of claim 1 for topical administration.

16. A method of treating, preventing or ameliorating a sulfur mustard skin injury in a subject comprising contacting the subject with a therapeutically effective amount of the formulation of claim 1.

17. The method of claim 16, wherein the contacting follows an exposure of the subject to sulfur mustard.

18. The method of claim 16, wherein the contacting follows immediately after the exposure of the subject to sulfur mustard.

19. The method of claim 16, wherein the contacting follows within about 1 minute to about 1 hour after the exposure of the subject to sulfur mustard.

20. The method of claim 19, wherein the contacting follows at the onset of symptoms of skin injury due to the exposure to sulfur mustard.

21. The method of claim 19, wherein the contacting is for at least 15 minutes.

22. A method of decontaminating a surface exposed to sulfur mustard comprising contacting the surface with the formulation of claim 1.

23. A method of preparing the formulation of claim 1, wherein the method comprises contacting sodium thiosulfate with a cellulose or a pharmaceutically acceptable salt thereof.

24. The method of claim 23, wherein the contacting is conducted in an aqueous solution.

25. The method of claim 23, wherein the contacting is conducted in a buffer.
